(19) United States
(12) Patent Application Publication
(10) Pub. No.: US 2018/0229005 A1
(43) Pub. Date: Aug. 16, 2018

(54) STABILIZATION OF TUBES PLACED IN THE BODY

(71) Applicants: TAO LIFE SCIENCES, Greenville, SC (US); GREENVILLE HOSPITAL SYSTEMS, Greenville, SC (US)

(72) Inventors: David E. Huizenga, Seneca, SC (US); John Chandler, Greenville, SC (US); Robert Gates, Greer, SC (US); Patrick Strane, Atlanta, GA (US); Mark McJunkin, Atlanta, GA (US)

(21) Appl. No.: 15/512,672

(22) PCT Filed: Sep. 18, 2015
(86) PCT No.: PCT/US15/50994

§ 371 Date: Mar. 20, 2017

Related U.S. Application Data
(60) Provisional application No. 62/053,030, filed on Sep. 19, 2014.

(51) Int. Cl. A61M 25/02; A61M 5/14

(52) U.S. Cl. CPC A61M 25/02; A61M 2025/0286; A61M 2025/028; A61M 5/1418

(57) ABSTRACT

Provided are devices for stabilizing a tube (e.g., a drainage tube such as a chest tube) in the body of a subject. The devices can comprise a base (102) configured to be secured to a patient comprising a patient contacting surface (104), an opposing top surface (106), a proximal end (108), a distal end (110), a tube-securing region (112), and a central reference plane (114); and one or more securement features (116, 118, 120) positioned within the tube-securing region. The one or more securement features (116, 118, 120) can be configured to reversibly engage the tube, such that when the tube is engaged by the one or more securement features (116, 118, 120), the tube is retained relative to the base (102).

### TECHNICAL FIELD

This application relates generally to devices for the stabilization of percutaneous tubes, as well as methods of using thereof.

### BACKGROUND

In many circumstances, tubes (e.g., percutaneous tubes), including drainage tubes, intravenous (IV) tubes, feeding tubes, or tracheal tubes, must be inserted into the body of a patient during the course of medical treatment. Once a tube is inserted in a patient, it can be advantageous to stabilize the tube (e.g., by attaching the tube to the patient's body) so as not to aggravate the insertion site or dislodge the tube from the body. Many means over the years have been used for such anchoring, including tape, sutures, weighted pressure, and a variety of clamping type devices (see, for example, U.S. Pat. Nos. 6,863,674, 3,683,911, 5,263,939, 6,238,373, 2,898,917, 6,592,573, 5,634,911, 5,279,575, 5,364,367, 5,217,441, 2,898,927, 5,620,424, 4,360,025, 4,672,979, 5,398,679, 5,803,079, 6,863,674, 7,811,293, and 3,487,837, and U.S. Patent Application Publication Nos. 2006/0025723, 2008/024308, 2007/0038177, and 2004/0106899).

For example, chest tubes are drainage tubes that are used to remove air (pneumothorax) or fluid (pleural effusion, blood, chyle), or pus (empyema) from the intrathoracic space. Chest tubes are frequently inserted after surgery or trauma sustained in the chest. Unfortunately, due to the nature of percutaneous tube insertion, complications often arise following placement of the tube. For example, up to 30% of chest tube insertions involve complications that can include partial or complete dislodgment of the tube. In the case of certain disease processes, such as tension pneumothorax, accidental dislodgment of the chest tube can be catastrophic. In addition, it can be extremely costly, difficult and painful to have to undertake a repeat insertion procedure at a new site following dislodgement.

Percutaneous tube securement generally utilizes adhesives often in combination with suturing. Unfortunately, the use of adhesives has been associated with increased risk of infection. When sutures are used, purse string suture or mattress suture closures are typically employed. In a purse string suture, a surgical suture is passed as a running stitch along the edge of the wound such that by drawing the two ends of the suture the wound is pulled around the tube. When utilizing a mattress stitch, the edges of the skin are pulled together tightly to form a smaller opening that abuts the tube wall. Unfortunately, these closures are complicated by the fact that they often fail to draw the tissue up tightly enough, leading to suture failure that leaves the tube unsecured. Sutures also must contain the proper amount of skin and subcutaneous tissue in order to be effective and secured properly. However, when used to secure a percutaneous tubes, these sutures must be placed near the cut edge of the skin where an appropriate amount skin and subcutaneous tissue is unavailable. In these confines, the sutures can frequently become unstable, leading to loss of hold of the percutaneous tube.

Improved method and devices for stabilizing percutaneous tubes, such as chest tubes, are needed so that, for example, tube movement is decreased, discomfort to the patient is decreased, and/or infections are decreased.

### SUMMARY

Provided are devices for stabilizing a tube in the body of a subject. The devices can comprise a base configured to be secured to a patient comprising a patient contacting surface, an opposing top surface, a proximal end, a distal end, a tube-securing region, and a central reference plane; and one or more securement features positioned within the tube-securing region. The one or more securement features can be configured to reversibly engage the tube, such that when the tube is engaged by the one or more securement features, the tube is retained relative to the base.

Optionally, the base can further comprise a tube inserting region. The tube-inserting region can comprise a first arm and a second arm that together at least partially define an aperture sized to permit passage of the tube through the aperture from a point above the top surface of the device to a point below the patient contacting surface of the device. In some embodiments, the first arm and a second arm together form an annular member that at least partially defines the aperture. The annular member can be a continuous annular member or a discontinuous annular member. In certain cases, the annular member can be a discontinuous annular member that includes an opening having a first dimension deformable to a second dimension. In these embodiments, the second dimension can be sized to permit passage of the tube through the opening of the annular member and into the aperture. In this way, the device can be readily interfaced with a percutaneous tube previously inserted into the body of a subject (e.g., by passing the tube into the aperture by way of the opening to position the aperture around the tube at the point of insertion). Optionally, the first dimension can be smaller than the outer diameter of the tube, so as to inhibit passage of the tube through the opening of the annular member when the opening is not deformed.

The devices can be secured to the body of a subject, and used to stabilize a percutaneous tube (e.g., a drainage tube such as a chest tube). In certain cases, the base can further comprise one or more anchor points (e.g., eyelets, hooks, or loops) that can be used to secure the device to the body of a subject. In some examples, the base can include a plurality of anchor points positioned symmetrically around the base.

In some cases, the base can comprise a tube inserting region that includes one or more anchor points. For example, in some embodiments, the device can include an annular member that comprises a plurality of anchor points. In certain embodiments, the device can include a plurality of anchor points positioned symmetrically around the annular member.

As described above, the device can include one or more securement features (e.g., prongs, clips, channels, or combinations thereof) positioned within the tube-securing region. The one or more securement features can be configured to reversibly engage the tube, such that when the tube is engaged by the one or more securement features, the tube is retained relative to the base. For example, in some cases, the one or more securement features can be configured such that when the tube is engaged by the one or more securement features, the one or more securement features apply at least 2 N (e.g., from 2 to 20 N) of securement force to the tube.

In certain embodiments, the one or more securement features can comprise a channel (e.g., a linear channel or a serpentine channel) comprising a tube contacting surface having an arcuate transverse cross-section. In some cases, the arcuate transverse cross-section of the tube contacting surface can have a radius of curvature equal to from 20% to 49% of the outer diameter of the tube. In some cases, the tube contacting surface can have a length of from 3 mm to 70 mm.

In some embodiments, the device can include at least two securement features positioned within the tube-securing region. For example, in some embodiments, the device can comprise a first securement feature positioned within the tube-securing region at a first location, and a second securement feature positioned within the tube-securing region at a second location spaced apart and distal to the first location.

In some embodiments, the first location, the second location, or both the first location and the second location are positioned along the central reference plane of the device. In other embodiments, the first location and the second location are positioned on opposite sides of the central reference plane. For example, the first location and the second location can be offset from one another (e.g., by an offset distance of from 1 mm to 15 mm) relative to the central reference plane by an offset distance, such that a vertically oriented reference plane disposed between the first location and the second location when the base is horizontally disposed is not parallel to or coplanar with the central reference plane.

In certain cases, the device can further include a third securement feature positioned within the tube-securing region at a third location spaced apart and distal to the second location. In some cases, the first location, the second location, and the third location can all be offset from one another relative to the central reference plane, such that vertically oriented reference planes disposed between the first location and the second location, the first location and the third location, and the second location and the third location when the base is horizontally disposed are not parallel to or coplanar with the central reference plane. In other cases, the second location can be offset from the first location and the third location relative to the central reference plane, such that vertically oriented reference planes disposed between the first location and the second location and the second location and the third location when the base is horizontally disposed are not parallel to or coplanar with the central reference plane, but a vertically oriented reference plane disposed between the first location the third location is parallel to or coplanar with the central reference plane.

In some embodiments, the one or more securement features can comprise a first prong comprising a first tube contacting surface upwardly projecting from the top surface of the base in the tube-securing region at a first location, and a second prong comprising a second tube contacting surface upwardly projecting from the top surface of the base in the tube-securing region at a second location spaced apart and distal to the first location. The first tube contacting surface and the second tube contacting surface can be exposed towards opposing sides of a vertically oriented reference plane disposed between the first location and the second location when the base is horizontally disposed. In certain cases, both the first tube contacting surface and the second tube contacting surface can possess an arcuate transverse cross-section. For example, a region of the first prong can extend horizontally over the base so as to form the first tube contacting surface having an arcuate transverse cross-section, and a region of the second prong can extend horizontally over the base so as to form the second tube contacting surface having an arcuate transverse cross-section. The region of the first prong and the region of the second prong can extend towards opposing sides of a vertically oriented reference plane disposed between the first location and the second location when the base is horizontally disposed. In certain embodiments, the region of the first prong and the region of the second prong can extend in substantially opposing directions. In certain embodiments, the arcuate transverse cross-section of the first tube contacting surface and the arcuate transverse cross-section of the second tube contacting surface can each have a radius of curvature equal to from 20% to 49% of the outer diameter of the tube. In certain embodiments, the first tube contacting surface and the second tube contacting surface can each have a length of from 3 mm to 70 mm (e.g., from 10 mm to 30 mm).

In some embodiments, the one or more securement features can further comprise a clip or channel projecting from the top surface of the base in the tube-securing region at a third location spaced apart and distal to the second location. The channel (e.g., a linear channel or a serpentine channel) can comprise a third tube contacting surface having an arcuate transverse cross-section. The arcuate transverse cross-section of the third tube contacting surface can have a radius of curvature equal to from 20% to 49% of the outer diameter of the tube. In some embodiments, the third tube contacting surface has a length of from 3 mm to 70 mm.

The devices described herein can be fabricated from any suitable material or combination of materials. In some embodiments, the device can be formed from a biocompatible material (e.g., a biocompatible silicone elastomer). In certain embodiments, the device can be formed from a silicone elastomer having a Shore A hardness of from 30 to 60, as measured by DIN 53505, a tensile strength of from 6 to 12 N/mm2 as measured by DIN 53504 S 1, a tear strength of from 20 N/mm to 70 N/mm, as measured by ASTM D624 B, or a combination thereof.

Also provided are methods of stabilizing a tube in the body of a subject using the devices provided herein.

### DESCRIPTION OF DRAWINGS

FIG. 1A is a perspective view of a device for stabilizing a tube in the body of a subject. The inset in FIG. 1A illustrates an enlargement of a securement feature of the device.

FIG. 1B is a bottom view of a device for stabilizing a tube in the body of a subject.

FIG. 1C is a top view of a device for stabilizing a tube in the body of a subject.

FIG. 2A is a perspective view of a device for stabilizing a tube in the body of a subject.

FIG. 2B is a top view of a device for stabilizing a tube in the body of a subject.

FIG. 2C is an additional perspective view of a device for stabilizing a tube in the body of a subject.

FIG. 3 is a perspective view of a device for stabilizing a tube in the body of a subject.

FIG. 4A is a perspective view of a device for stabilizing a tube in the body of a subject. An inset illustrates an enlargement of the tube-securing region of the device.

FIG. 4B is an additional perspective view of a device for stabilizing a tube in the body of a subject.

FIG. 5A is a perspective view of a device for stabilizing a tube in the body of a subject.

FIG. 5B is an additional perspective view of a device for stabilizing a tube in the body of a subject.

FIG. 6A is a perspective view of a device for stabilizing a tube in the body of a subject. An inset illustrates an enlargement of the tube-securing region of the device.

FIG. 6B is an additional perspective view of a device for stabilizing a tube in the body of a subject.

FIG. 7A is a perspective view of a device for stabilizing a tube in the body of a subject.

FIG. 7B is a longitudinal cross-sectional view of a device for stabilizing a tube in the body of a subject.

FIG. 7C is a longitudinal cross-sectional view of a device for stabilizing a tube in the body of a subject.

FIG. 8 is an illustration of the transverse cross-section of eight securement features having berms of varying widths. The width of the berm of the securement features increases from the top securement feature to the bottom securement feature. The inset in FIG. 8 illustrates an enlargement of a representative securement feature.

FIG. 9 is a top view of the device illustrated in FIG. 2C. Certain dimensions are noted in mm.

FIG. 10 is a top view of the device illustrated in FIGS. 4A-B. Certain dimensions are noted in mm.

FIG. 11 is a top view of the device illustrated in FIGS. 5A-B. Certain dimensions are noted in mm.

FIG. 12 is a top view of a device for stabilizing a tube in the body of a subject. Certain dimensions are noted in mm.

FIG. 13 is a top view of a device illustrated in FIG. 7C. Certain dimensions are noted in mm.

FIGS. 14A-14B illustrate a mold used to prepare a jig containing example securement features having tube contacting surfaces of varying lengths.

FIGS. 15A-15B illustrate a mold used to prepare a jig containing example securement features having arcuate tube contacting surfaces with varying radii of curvature.

FIGS. 16A-16B illustrate a mold used to prepare a jig containing example securement features with varying berms.

FIG. 17A is a plot showing the relationship between the length of the tube contacting surface of a securement feature and the securement force produced by the securement feature. The securement features tested were fabricated from materials having a Shore A hardness of 40.

FIG. 17B is a plot showing the relationship between the length of the tube contacting surface of a securement feature and the securement force produced by the securement feature. The securement features tested were fabricated from materials having a Shore A hardness of 50.

FIG. 17C is a plot showing the relationship between the length of the tube contacting surface of a securement feature and the securement force produced by the securement feature. The securement features tested were fabricated from materials having a Shore A hardness of 60.

FIG. 18 is a plot showing a comparison the of results illustrated in FIGS. 17A-C. Best-fit lines for each data set are superimposed on the plot.

FIG. 19 is a bar graph plotting the slopes of the best-fit lines included in FIG. 18.

FIG. 20 is a plot showing the relationship between the diameter of the tube contacting surface of a securement feature and the securement force produced by the securement feature.

FIG. 21A is a plot of the securement force applied by a securement feature versus the diameter of the securement feature for example securement features having a diameter of 60-80% of the diameter of the tubing being secured. Best-fit lines for each data set are superimposed on the plot.

FIG. 21B is a plot of the securement force applied by a securement feature versus the diameter of the securement feature for example securement features having a diameter of 80-100% of the diameter of the tubing being secured. Best-fit lines for each data set are superimposed on the plot.

FIG. 22A is a bar graph plotting the slopes of the best-fit lines included in FIG. 21A.

FIG. 22B is a bar graph plotting the slopes of the best-fit lines included in FIG. 21B.

FIG. 23 is a photograph illustrating a device for stabilizing a tube in the body of a subject engaged with a tube.

FIG. 24 is a plot of the securement force applied by a securement feature versus the berm of the securement feature for example securement features having various berms. Best-fit non-linear curves for each data set obtained using a modified Michaelis-Menten-type analysis are superimposed on the plot

FIG. 25A is a bar graph illustrating the relationship between securement force and berm.

FIG. 25B is a bar graph illustrating the relationship between securement force and berm.

### DETAILED DESCRIPTION

Reference will now be made in detail to various embodiments of the disclosed subject matter, one or more examples of which are set forth below. Each embodiment is provided by way of explanation of the subject matter, not a limitation of the subject matter. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment. Thus, it is intended that the present disclosure cover such modifications and variations as come within the scope of the appended claims and their equivalents.

The present disclosure is generally directed to stabilization devices for percutaneous tubes. More specifically, the disclosed devices can be utilized to properly align and secure a percutaneous tube following implantation. In some embodiments, the stabilization devices can be utilized to properly align and secure a thoracostomy tube within the pleural cavity. Tube thoracostomy is a procedure that is used to drain the pleural space of air, mucus, blood, or any other fluid. It should be understood, however, that while the disclosed stabilization devices may prove beneficial when utilized in conjunction with a thoracostomy tube, the disclosed devices are in no way limited to utilization with chest tubes, and the device may be utilized to stabilize any percutaneous tube including, without limitation, surgical drainage tubes, gastronomy tubes, Y-shaped cardiac drainage systems, vascular access tubes, central lines, venous and arterial access ports, colostomy tubes, and so forth. In certain embodiments, the tube can be a 6 French (Fr), 7 Fr, 8 Fr, 9 Fr, 10 Fr, 11 Fr, 12 Fr, 13 Fr, 14 Fr, 15 Fr, 16 Fr, 17 Fr, 18 Fr, 19 Fr, 20 Fr, 21 Fr, 22 Fr, 23 Fr, 24 Fr, 25 Fr, 26 Fr, 27 Fr, 28 Fr, 29 Fr, 30 Fr, 31 Fr, 32 Fr, 33 Fr, 34 Fr, 35 Fr, 36 Fr, 37 Fr, 38 Fr, 39 Fr, or 40 Fr tube. In certain embodiments, the tube can be a 20 Fr to 40 Fr tube.

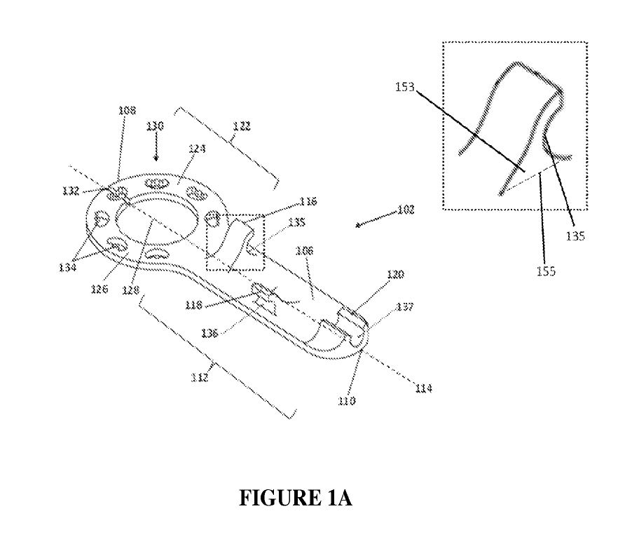

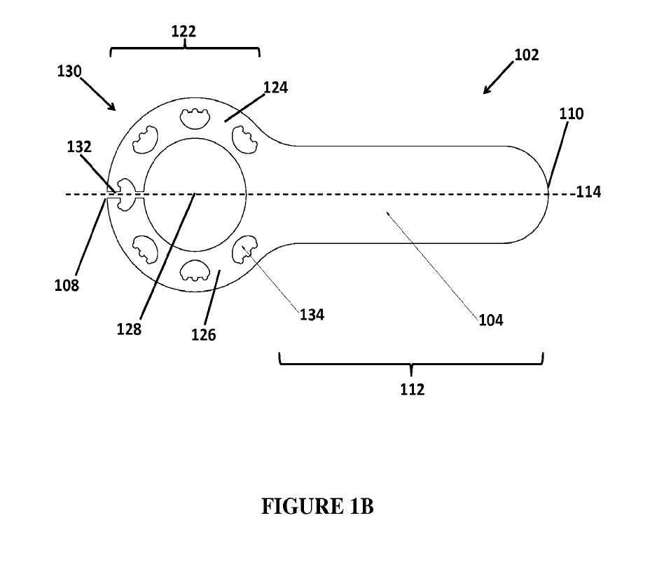

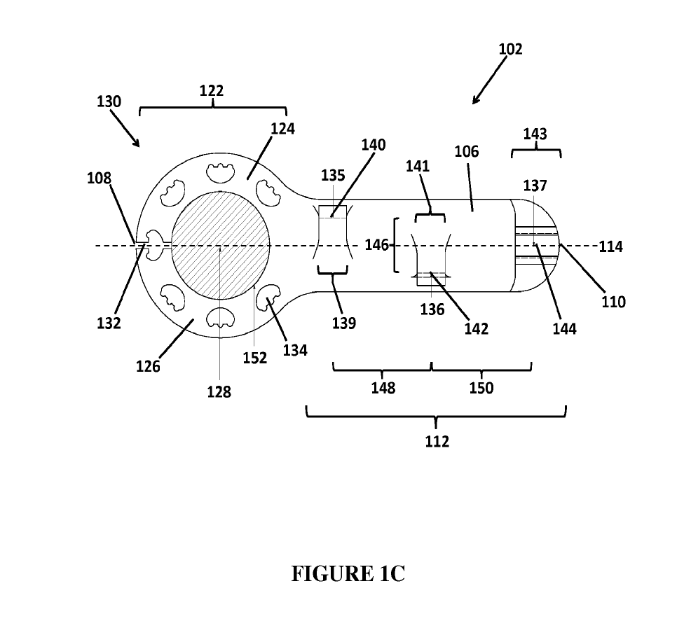

Referring now to FIGS. 1A-1C, devices for stabilizing a tube in the in the body of a subject can comprise a base (102) configured to be secured to a patient. The base (102) can comprise a patient contacting surface (104), an opposing top surface (106), a proximal end (108), a distal end (110), a tube-securing region (112), and a vertically oriented central reference plane (114) running from the proximal end of the base (108) to the distal end of the base (110) when the base is horizontally disposed. The devices can further include one or more securement features (116, 118, 120) positioned within the tube-securing region (112). The one or more securement features can be configured to reversibly engage the tube, such that when the tube is engaged by the one or more securement features, the tube is retained relative to the base.

The device can have any suitable shape or size, provided that the size and shape are compatible with placement of the device on the subject. The footprint of the device can be generally rectangular or ovoid. Alternatively, the footprint of the device can be generally circular, square, trapezoidal, or polygonal in shape. As utilized herein the term ‘footprint’ is intended to refer to the overall shape of the device over that surface of the base that is in contact with the subject during use.

The dimensions of the footprint can generally vary depending upon the specific application, e.g., the anchoring site, the expected duration of the insertion, subject size, tube size, etc. For example, the footprint of the device can have a total area contacting the subject of less than or equal to, 2000 mm², 1800 mm², 1600 mm², 1400 mm², 1200 mm², 1000 mm², 900 mm², 800 mm², 700 mm², 600 mm², 500 mm², 400 mm², 300 mm², or 200 mm². In one embodiment, the base can be specifically designed for a particular anchoring location on the body of the subject. In these embodiments, the footprint of the device can be such that the device fits at that location.

Devices for stabilizing a tube in the in the body of a subject can include one or more securement features positioned within the tube-securing region. The structure and dimensions of each of the one or more securement features in the device can vary to provide the desired securement force to the tube. For example, each of the one or more securement features can individually be, for example, a channel (e.g., a linear channel or a serpentine channel), a prong, or a clip as discussed in more detail below. Each of the one or more securement features can include a tube contacting surface which, through pressure and friction, can provide a securing force to a tube positioned in contact with the tube contacting surface of the securement features (i.e., a tube “engaged” by the securement feature).

For example, in some embodiments, the tube contacting surface can possess an arcuate transverse cross-section. In some cases, the dimensions of the tube contacting surface (e.g., a radius of curvature of the arcuate transverse cross-section) can be selected such that the tube contacting surface exhibits an interference fit with the tube. For example, in some cases, the tube contacting surface can have an arcuate transverse cross-section having a radius of curvature that is smaller than 50% of the outer diameter of the tube (i.e., smaller than the radius of the tube).

For example, the arcuate transverse cross-section of the tube contacting surface can have a radius of curvature of less than 49% of the outer diameter of the tube (e.g., less than 48%, less than 47%, less than 46%, less than 45%, less than 44%, less than 43%, less than 42%, less than 41%, less than 40%, less than 39%, less than 38%, less than 37%, less than 36%, less than 35%, less than 34%, less than 33%, less than 32%, less than 31%, less than 30%, less than 29%, less than 28%, less than 27%, less than 26%, less than 25%, less than 24%, less than 23%, less than 22%, less than 41%, or less). In some embodiments, the arcuate transverse cross-section of the tube contacting surface can have a radius of curvature of at least 20% of the outer diameter of the tube (e.g., at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, or at least 49%).

The arcuate transverse cross-section of the tube contacting surface can have a radius of curvature ranging from any of the minimum values described above to any of the maximum values described above. For example, the arcuate transverse cross-section of the tube contacting surface can have a radius of curvature of from 20% to 49% of the outer diameter of the tube (e.g., from 35% to 45%).

Generally, a tube contacting surface having an arcuate transverse cross-section with a smaller radius of curvature relative to the radius of the tube will apply more securement force to the tube when the tube is engaged. However, it should be appreciated that this is influenced by the material used to fabricate the securement feature, as well as the overall design of the securement feature. For example, in the case of securement features formed from relatively hard materials (e.g., a near plastic such as a Shore D 50-80 material), the securement feature accommodates for a relatively small deformation of the tube contacting surface around the tube upon engagement of the tube in the securement feature. In the case of securement features formed from softer materials (e.g., a Shore D 30-50 material), the securement feature accommodates more deformation of the tube contacting surface around the tube upon engagement of the tube in the securement feature. As such, a securement feature fabricated from relatively hard material can be expected to apply a larger securement force to an engaged tube, relative to securement feature having the same dimensions fabricated from a softer material.

The tube contacting surface can be formed so as to contact varying amounts of the surface of the tube at a given point along the tube contacting surface. For example, in the case of a tube contacting surface having an arcuate transverse cross-section, the angle subtended by the arcuate transverse cross-section of the tube contacting surface can be at least 90, 100, 110, 120, 130, 140, 150, 160, 170, 180, 190, 200, 210, 220, 230, 240, 250, 260, 270, 280, 290, 300, 310, 320, 330, 340, or 350 degrees.

The length of the tube contacting surface can vary. Generally, a longer tube contacting surface can be expected to apply a larger securement force to an engaged tube. In some cases, the length of the tube contacting surface can be 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, 50 mm, 51 mm, 52 mm, 53 mm, 54 mm, 55 mm, 56 mm, 57 mm, 58 mm, 59 mm, 60 mm, 61 mm, 62 mm, 63 mm, 64 mm, 65 mm, 66 mm, 67 mm, 68 mm, 69 mm, 70 mm, 71 mm, 72 mm, 73 mm, 74 mm, 75 mm, 76 mm, 77 mm, 78 mm, 79 mm, 80 mm, 81 mm, 82 mm, 83 mm, 84 mm, 85 mm, 86 mm, 87 mm, 88 mm, 89 mm, 90 mm, 91 mm, 92 mm, 93 mm, 94 mm, 95 mm, 96 mm, 97 mm, 98 mm, 99 mm, or 100 mm. The length of the tube contacting surface can range between any of the values above. For example, the length of the tube contacting surface can be from 1 mm to 100 mm (e.g., from 5 mm to 75 mm, from 3 mm to 70 mm, from 10 mm to 30 mm, or from 3 mm to 12 mm). In certain embodiments, the length of the tube contacting surface can be from 3 mm to 70 mm (e.g., from 10 mm to 30 mm).

The tube-contacting surfaces of the securement features can be smooth. Alternatively, in some embodiments, the tube-contacting surfaces of the securement features can include textural features (e.g., ridges or protrusions) to influence the securement force applied to the tube by the securement feature. See, for example, WO 2014/031860 to Clemson University Research Foundation and Greenville Hospital System, which is hereby incorporated by reference in its entirety. Optionally, the tube-contacting surfaces of the securement features can include, for example, an adhesive material (e.g., a pressure-sensitive adhesive deposited on the tube-contacting surface) to facilitate retention of the tube relative to the base when the tube is engaged by the securement feature.

Optionally, securement features can include flaps or other features that can be used to facilitate retention of the tube relative to the base when the tube is engaged by the securement feature. For example, in certain embodiments, the securement features can include one or more hooks or loops that can be used to clasp or secure one point on the securement feature to another point on the securement feature or another point on the device. For example, the securement feature can include a hook or loop that can be used as an anchor point that can be sutured, alone or in conjunction with another anchor point on the device, to further secure in contact with the tube contacting surface of the securement feature.

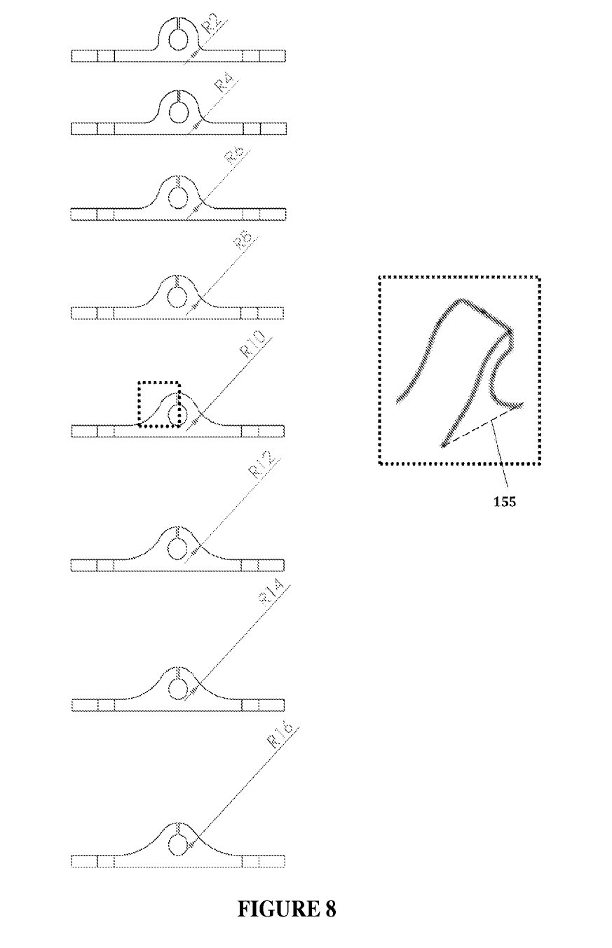

The structure of the securement feature can be varied to tailor the amount of securement force exerted by the securement feature on a tube engaged by the securement feature. For example, in some embodiments, as illustrated in the inset shown in FIG. 1A, a securement feature can include a tube contacting surface (135) supported by a berm (153). As illustrated in FIG. 8, securement features can possess berms of varying width. For example, the berm can be of varying width (155), measured as a distance along a plane parallel to the top surface of the base (106). For example, the width of the berm can be 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, or 30 mm. The width of the berm can range between any of the values above. For example, the width of the berm can be from 1 mm to 30 mm (e.g., from 1 mm to 15 mm, or from 1 mm to 10 mm). In some embodiments, the width of the berm can be at least 50% of the thickness of the base (102) (i.e., the distance between the patient contacting surface of the base (104) and the top surface of the base (106)).

The width of the berm can influence the overall rigidity of the securement feature. In general, an increase in the width of the berm in a securement feature will increase the rigidity of the securement feature. As a consequence, the securement feature will generally be less susceptible to deformation, and exert a greater securement force on a tube engaged by the securement feature.

The number of securement features positioned within the tube-securing region of the device can be varied, for example, depending on the nature of the tube being secured, the design of the one or more securement features present in the device, and the relative orientation of the one or more securement features in the device. The number of securement features, the design of the one or more securement features, and the relative positioning of the one or more securement features can be selected such that when the tube is engaged by the one or more securement features, the tube is retained relative to the base (e.g., such that it remains substantially immobile relative to the base). For instance, a device can include 1, 2, 3, 4, 5, 6, 7, 8, 9, 10, or more securement features. In some embodiments, the device can include a single securement feature positioned within the tube-securing region. In other embodiments, the device can include from one to four securement feature positioned within the tube-securing region.

In some embodiments, the device can include at least two securement features positioned within the tube-securing region. For example, the device can comprise a first securement feature positioned within the tube-securing region at a first location, and a second securement feature positioned within the tube-securing region at a second location spaced apart and distal to the first location.

By way of example, referring now to FIGS. 1A-1C, the device can comprise a first securement feature (116) positioned within the tube-securing region (112) at a first location (140), and a second securement feature (118) positioned within the tube-securing region (112) at a second location (142) spaced apart and distal to the first location.

The distance from the first location to the second location (148), as measured along the central reference plane of the device (114), can vary. For example, the first location and the second location can be separated by a distance, as measured along the central reference plane of the device, of 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, 50 mm, 51 mm, 52 mm, 53 mm, 54 mm, 55 mm, 56 mm, 57 mm, 58 mm, 59 mm, 60 mm, 61 mm, 62 mm, 63 mm, 64 mm, 65 mm, 66 mm, 67 mm, 68 mm, 69 mm, 70 mm, 71 mm, 72 mm, 73 mm, 74 mm, 75 mm, 76 mm, 77 mm, 78 mm, 79 mm, 80 mm, 81 mm, 82 mm, 83 mm, 84 mm, 85 mm, 86 mm, 87 mm, 88 mm, 89 mm, 90 mm, 91 mm, 92 mm, 93 mm, 94 mm, 95 mm, 96 mm, 97 mm, 98 mm, 99 mm, or 100 mm. The distance can range between any of the values above. For example, the first location and the second location can be separated by a distance, as measured along the central reference plane of the device, of from 1 mm to 100 mm (e.g., from 5 mm to 75 mm, from 3 mm to 70 mm, from 10 mm to 30 mm, or from 3 mm to 12 mm). In certain embodiments, the first location and the second location can be separated by a distance, as measured along the central reference plane of the device, of from 3 mm to 70 mm (e.g., from 10 mm to 30 mm).

In some embodiments, the first location, the second location, or both the first location and the second location can be positioned along the central reference plane of the device.

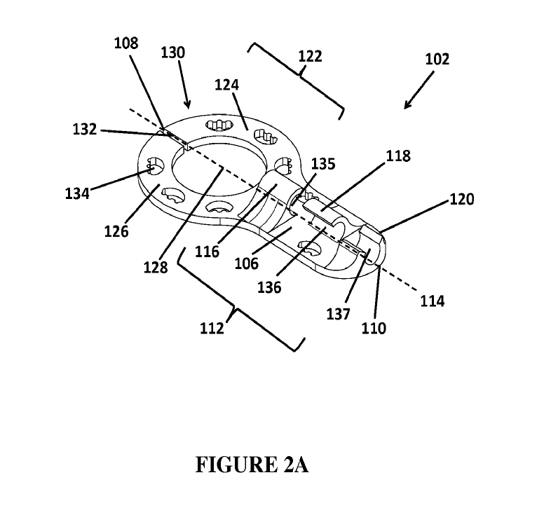

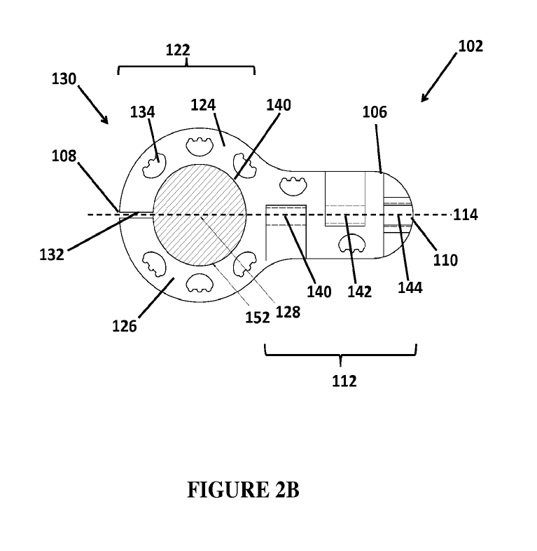

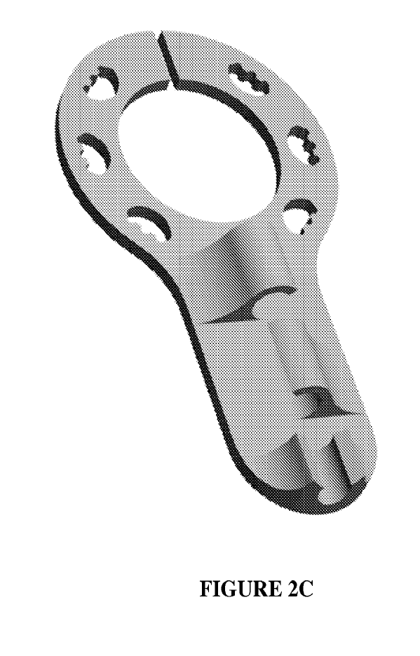

In some cases, both the first location and the second location can be positioned along the central reference plane of the device. By way of example, referring now to FIGS. 2A-2B, the device can comprise a first securement feature (116) positioned within the tube-securing region (112) at a first location (140), and a second securement feature (118) positioned within the tube-securing region (112) at a second location (142) spaced apart and distal to the first location. Both the first location (140) and the second location (142) can be positioned along the central reference plane of the device (114). In this case, the first securement feature (116) and the second securement feature (118) can be said to be aligned, as a vertically oriented reference plane disposed between the first location and the second location when the base is horizontally disposed is parallel to or coplanar with the central reference plane.

In other embodiments, the first location and the second location are positioned on opposite sides of the central reference plane. For example, the first location and the second location can be offset from one another relative to the central reference plane by an offset distance, such that a vertically oriented reference plane disposed between the first location and the second location when the base is horizontally disposed is not parallel to or coplanar with the central reference plane.

By way of example, referring now to FIGS. 1A-1C, the device can comprise a first securement feature (116) positioned within the tube-securing region (112) at a first location (140), and a second securement feature (118) positioned within the tube-securing region (112) at a second location (142) spaced apart and distal to the first location. The first location (140) and the second location (142) can be positioned on opposite sides of the central reference plane of the device (114). In this case, the first securement feature (116) and the second securement feature (118) are offset from one another relative to the central reference plane (114) by an offset distance (146), such that a vertically oriented reference plane disposed between the first location (140) and the second location (142) when the base is horizontally disposed is not parallel to or coplanar with the central reference plane (114).

The offset distance (146) between the first location and the second location can vary. For example, the offset distance can be 1 mm, 2 mm, 3 mm, 4 mm, 5 mm, 6 mm, 7 mm, 8 mm, 9 mm, 10 mm, 11 mm, 12 mm, 13 mm, 14 mm, or 15 mm. The offset distance can range between any of the values above. For example, the offset distance can be from 1 mm to 15 mm (e.g., from 2 mm to 5 mm, from 3 mm to 8 mm, or from 9 mm to 15 mm).

In these embodiments, the offset of the first location and the second location provides a tortuous path for a tube engaged with securement features positioned at the first location and the second location. An offset arrangement of multiple securement features so as to provide a tortuous path for a tube engaged with the securement features is referred to herein as a “swale” or “swaling.” When the tube is engaged by the offset securement features, the tube is snaked or bent around the securement features. When a force is applied to the engaged tube distal to the device, the tube can be pulled against a tube contacting surface of a first securement feature relative to the tube contacting surface of an adjacent offset securement feature, resulting in an additional securement force being applied to the engaged tube. In addition, swaling can provide additional dissipating vector actions when a force is applied to the tube distal to the device, helping to decrease movement of the tube at the insertion site when a force or movement is applied to the tube away from the tube insertion site. The devices can include more than two offset or swaled securement features, as discussed in more detail below.

In certain embodiments, the device can include at least three securement features positioned within the tube-securing region. For example, the device can comprise a first securement feature positioned within the tube-securing region at a first location, a second securement feature positioned within the tube-securing region at a second location spaced apart and distal to the first location, and a third securement feature positioned within the tube-securing region at a third location spaced apart and distal to the second location.

By way of example, referring again to FIGS. 1A-1C, the device can include a first securement feature (116) positioned within the tube-securing region (112) at a first location (140), a second securement feature (118) positioned within the tube-securing region (112) at a second location (142) spaced apart and distal to the first location, and a third securement feature (120) positioned within the tube-securing region (120) at a third location (144) spaced apart and distal to the second location (142).

The distance from the second location to the third location (150), as measured along the central reference plane of the device (114), can vary. For example, the second location and the third location can be separated by a distance, as measured along the central reference plane of the device, of 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, 50 mm, 51 mm, 52 mm, 53 mm, 54 mm, 55 mm, 56 mm, 57 mm, 58 mm, 59 mm, 60 mm, 61 mm, 62 mm, 63 mm, 64 mm, 65 mm, 66 mm, 67 mm, 68 mm, 69 mm, 70 mm, 71 mm, 72 mm, 73 mm, 74 mm, 75 mm, 76 mm, 77 mm, 78 mm, 79 mm, 80 mm, 81 mm, 82 mm, 83 mm, 84 mm, 85 mm, 86 mm, 87 mm, 88 mm, 89 mm, 90 mm, 91 mm, 92 mm, 93 mm, 94 mm, 95 mm, 96 mm, 97 mm, 98 mm, 99 mm, or 100 mm. The distance can range between any of the values above. For example, the second location and the third location can be separated by a distance, as measured along the central reference plane of the device, of from 1 mm to 100 mm (e.g., from 5 mm to 75 mm, from 3 mm to 70 mm, from 10 mm to 30 mm, or from 3 mm to 12 mm). In certain embodiments, the second location and the third location can be separated by a distance, as measured along the central reference plane of the device, of from 3 mm to 70 mm (e.g., from 10 mm to 30 mm).

In some cases, as illustrated in FIGS. 1A-1C, the first location (140), the second location (142), and the third location (144) can all be offset or swaled with respect to one another, such that vertically oriented reference planes disposed between the first location and the second location, the first location and the third location, and the second location and the third location when the base is horizontally disposed are not parallel to or coplanar with the central reference plane (114).

In other embodiments, as illustrated in FIGS. 2A-2B, the first location (140), the second location (142), and the third location (144) can all be positioned along the central reference plane of the device (114).

In other embodiments, the second location can be offset from the first location and the third location relative to the central reference plane, such that vertically oriented reference planes disposed between the first location and the second location and the second location and the third location when the base is horizontally disposed are not parallel to or coplanar with the central reference plane, but a vertically oriented reference plane disposed between the first location the third location is parallel to or coplanar with the central reference plane.

As described above, the one or more securement features, in combination, can be configured to reversibly engage the tube, such that when the tube is engaged by the one or more securement features, the tube is retained relative to the base. For example, the one or more securement features can be configured such that when the tube is engaged by the one or more securement features, the one or more securement features apply a securement force effective to provide for retention of the tube relative to the base (e.g., in conjunction with normal use to secure a tube in a subject).

In some embodiments, the one or more securement features can be configured such that when the tube is engaged by the one or more securement features, the one or more securement features apply at least 2 N (e.g., at least 3 N, at least 4 N, at least 5 N, at least 6 N, at least 7 N, at least 8 N, at least 9 N, at least 10 N, at least 12 N, at least 13 N, at least 14 N, at least 15 N, at least 16 N, at least 17 N, at least 18 N, at least 19 N, or more) of securement force to the tube. In some embodiments, the one or more securement features can be configured such that when the tube is engaged by the one or more securement features, the one or more securement features apply 20 N of securement force to the tube or less (e.g., 19 N or less, 18 N or less, 17 N or less, 16 N or less, 15 N or less, 14 N or less, 13 N or less, 12 N or less, 11 N or less, 10 N or less, 9 N or less, 8 N or less, 7 N or less, 6 N or less, 5 N or less, 4 N or less, or 3 N or less).

The one or more securement features can be configured such that when the tube is engaged by the one or more securement features, the one or more securement features apply a securement force to the tube ranging from any of the minimum values described above to any of the maximum values described above. For example, the one or more securement features are configured such that when the tube is engaged by the one or more securement features, the one or more securement features apply from 2 N to 20 N (e.g., from 2 N to 15 N) of securement force to the tube.

The securement force exerted by one or more securement features can be determined using standard methods known in the art. For example, the securement force can be determined by applying a force to a tube engaged by the one or more securement features in a given direction, and measuring the amount of force required to elicit movement of the tube relative to the base in the direction of the applied force (e.g., sliding of the tube through the one or more securement features along an axis generally parallel to the central reference plane upon application of force to the tube along that same axis, or the tube breaking away from one or more of the securement features upon application of force to the tube along an axis generally perpendicular to the base of the device). The applied force at which movement of the tube relative to the base begins is equal to securement force exerted by the one or more securement features on the engaged tube.

Optionally, as in the example device illustrated in FIGS. 1A-1C, the base can further comprise a tube inserting region (122). When present in the device, the tube inserting region can help a health care practitioner properly locate and secure the device in the appropriate region of the body relative to the tube insertion site on the body. In addition, the tube inserting region can be configured to increase the stability of the tube and/or decrease movement of the tube at the insertion site, through for example, vectoring and distribution of forces away from the insertion site when forces or movement occur on the tube. In some cases, the tube inserting region can be absent from the device. For example, the proximal end of the base can terminate in a blunt end beyond which the tube is inserted.

When present, the tube inserting region can adopt a variety of shapes. Referring to FIG. 1A, is some cases, the tube-inserting region (122) can comprise a first arm (124) and a second arm (126). The first arm (124) and the second arm (126) can together at least partially define an aperture (128) sized to permit passage of the tube through the aperture (128) from a point above the top surface of the device to a point below the patient contacting surface of the device.

As illustrated in FIGS. 1A-1C, in some embodiments, the first arm (124) and the second arm (126) together form an annular member (130) that at least partially defines the aperture (128). The annular member (130) can be a discontinuous annular member that includes one or more openings (132) along its circumference, as in the example device illustrated in FIGS. 1A-1C. Alternatively, the annular member can be a continuous annular member that does not include one or more openings along its circumference. The dimensions of opening (132) can vary. In certain cases, the opening has a first dimension deformable to a second dimension. In these embodiments, the second dimension can be sized to permit passage of the tube through the opening of the annular member and into the aperture. In this way, the device can be readily interfaced with a percutaneous tube previously inserted into the body of a subject. For example, a percutaneous tube previously inserted into the body of a subject can be passed into the aperture (128) by way of the opening (132) in order to position the aperture (128) over the tube insertion site such that the annular member (130) is circumferentially disposed around the tube insertion site. In this position, the tube can pass from the patient's body through the aperture (128) to a point above the top surface of the device (106). The tube can then be engaged by the one or more securement features (116, 118, 120) positioned within the tube-securing region (112), such that the tube is retained relative to the base (102). Optionally, the first dimension of the opening (132) can be smaller than the outer diameter of the tube, so as to inhibit passage of the tube through the opening (132) of the annular member (130) when the opening (132) is not deformed.

The dimensions (size and shape) of the aperture can vary depending upon the specific application for the device (e.g., the desired anchoring site, the expected duration of the insertion, tube size, etc.). In some cases, as illustrated in FIGS. 1A-1C, the aperture can have a generally circular or ellipsoid shape. Alternatively, the aperture can be generally rectangular, square, trapezoidal, or polygonal in shape. In certain embodiments, the aperture can be a generally circular or ellipsoid opening defined by an interior wall (152) having a diameter of 2 mm, 3 mm, 4 mm, 5 mm, 6 mm, 7 mm, 8 mm, 9 mm, 10 mm, 11 mm, 12 mm, 13 mm, 14 mm, 15 mm, 20 mm, 25 mm, 30 mm, 35 mm, 40 mm, 45 mm, 50 mm, 55 mm, 60 mm, 65 mm, 70 mm, or values between these. In some embodiment, the aperture can be a generally circular or ellipsoid opening defined by an interior wall (152) having a diameter of from 5 mm to 50 mm (e.g., from 10 mm to 40 mm).

The devices can be secured to the body of a subject, and used to stabilize a percutaneous tube (e.g., a drainage tube such as a chest tube). The devices can be secured to the subject using any suitable method (e.g., via suturing, the use of an adhesive, or a combination thereof). In some cases, the stabilization device can be configured to be secured to the body of a subject using sutures alone (e.g., the device can be suitably secured to the subject without the use of an adhesive in conjunction with the sutures). In certain cases, the stabilization device can be configured to be secured to the body of a subject using fewer sutures than have been utilized for the suture securement of percutaneous tubes in the past (e.g., a mattress suture or purse string suture technique). As such, use of the device can reduce complications associated with sutures such as risk of air leakage, skin necrosis, and poor cosmetic results. Adhesives can cause irritation as well as create a potential infection at the insertion site. Thus, elimination of the need for adhesives can increase the safety of the insertion process.

The devices can optionally include features to facilitate securement of the device to the body of a subject using one or more sutures. Referring again to FIGS. 1A-1C, in some embodiments, the base can further comprise one or more anchor points (134). As illustrated in FIGS. 1A-1C, the anchor points (134) can be eyelets that are used to anchor the stabilization device to the skin surface at the site of tube insertion. For example, the device can be sutured to the skin of the subject using the series of anchor points (eyelets, 134) that are located along the perimeter of the base (102). The eyelets permit passage of a suture through the base of the device and through the skin.

Optionally, as illustrated in FIGS. 1A-1C, the eyelets can include a series of insets that provide distinct resting sites for the sutures. During anchoring of the device, the suture can be set within an inset. The insets can have rounded edges, which can eliminate sharp corners that can become areas of high stress concentration. In addition, through location of the suture within an inset, relative motion between the suture and the base will be decreased and the device will be held more firmly, preventing any rotation that can lead to dislodgement. Optionally, the eyelets can be, for example, filed with a polymer membrane which can be pierced during attachment of the sutures. Alternatively, the anchor points can be hooks or loops around which or through which sutures are passed to secure the device.

Though the device illustrated in FIGS. 1A-1C includes seven anchor points, the device can include any number of anchor points. For instance, a device may have 2, 3, 4, 5, 6, 7, 8, 9, 10, or more anchor points. In some embodiments in which the device includes a large number of anchor points, for instance more than five anchor points, such as the seven anchor points illustrated in FIGS. 1A-1C, a surgeon can have increased flexibility to use as many or as few of the anchor points as is necessary to properly secure the device to the skin.

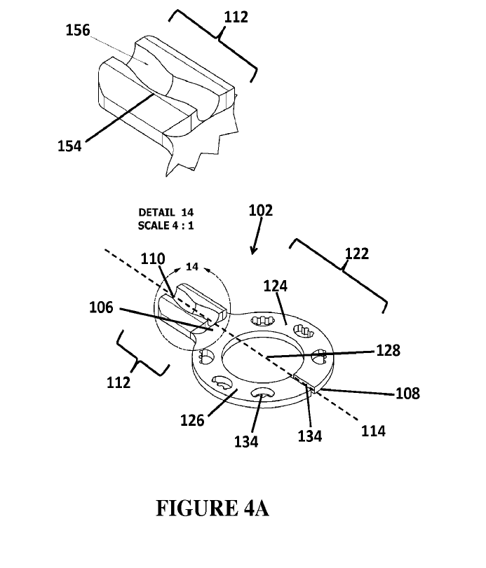

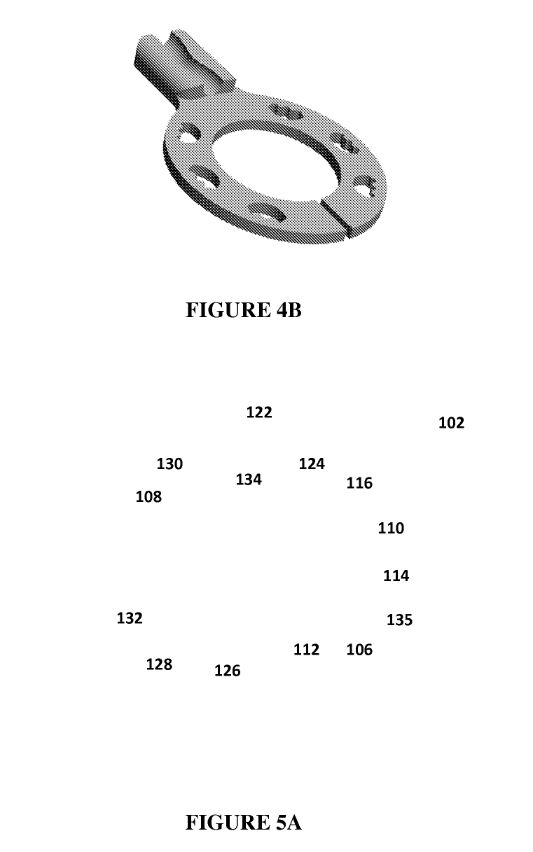

Optionally, the base portion further comprises a plurality of anchor points. Optionally, the anchor points are positioned symmetrically around the base portion, as in the example devices illustrated in FIGS. 4, 5, and 6.

As described above, devices for stabilizing a tube in the in the body of a subject can optionally include a tube inserting region. Optionally, as illustrated in FIGS. 1A-1C, the device can include one or more anchor points (134) positioned in the tube inserting region (122). For example, in some embodiments, the device can include an annular member (130) that comprises a plurality of anchor points (130). In certain embodiments, the device can include a plurality of anchor points (134) positioned symmetrically around the annular member (130). As described above, when devices of this type care employed to stabilize a percutaneous tube, the aperture (128) can be positioned over the tube insertion site, such that the annular member (130) is circumferentially disposed around the tube insertion site. The annular member (130) including a plurality of anchor points (134) can thus be used as a suture ring to secure the device to the subject.

In these embodiments, the dimensions of the aperture (128) and the annular member (130), as well as the positioning of the plurality of anchor points (134) around the annular member (130) can be selected such that the plurality of anchor points can be located at a distance from the tube insertion site of at least 5 mm (e.g., at least 10 mm, or more). This arrangement can provides for suturing of the device to skin at a distance from the incision formed for the insertion. This can further improve the stability of the tube, as the suture sites can be less likely to pull-out as can happen when the sutures are very near the incisions (as is the case for purse string and mattress sutures), as the tissue very near the insertion site can be more easily subjected to degradation due to infection and necrosis. In addition, sutures very near the incision can be subjected to additional tube pressure, and movement of the tube can cause trauma within the body as well as to the surrounding subcutaneous tissue via the sutures, thus causing bruising, hematoma, etc.

Optionally, an adhesive can be used to secure the device. In these embodiments, a biocompatible adhesive can be disposed on the patient contacting surface of the device. Optionally, an anti-infective agent (e.g., a silver-, gold-, copper-, or zinc-containing compound, a as a silver impregnated alginate) can be disposed on the patient contacting surface of the device to prevent infection.

Certain example embodiments are discussed in more detail below to further illustrate aspects of the devices described herein.

Referring now to FIGS. 1A-1C, in some embodiments, devices for stabilizing a tube in the body of a subject can include a base (102) comprising a patient contacting surface (104), an opposing top surface (106), a proximal end (108), a distal end (110), a tube-securing region (112), and a vertically oriented central reference plane (114) running from the proximal end of the base (108) to the distal end of the base (110) when the base is horizontally disposed. The devices can further include three securement features (116, 118, and 120) positioned within the tube-securing region (112). The three securement features can be configured to reversibly engage the tube, such that when the tube is engaged by the three securement features, the tube is retained relative to the base.

As illustrated in FIGS. 1A-1C, in some embodiments, the device can include a first securement feature (116; a prong) comprising a first tube contacting surface (135) upwardly projecting from the top surface of the base (106) in the tube-securing region (112) at a first location (140), and a second securement feature (118; a prong) comprising a second tube contacting surface (136) upwardly projecting from the top surface of the base (106) in the tube-securing region at a second location (142) spaced apart (e.g., by distance 148) and distal to the first location (140). The tube contacting surfaces of the first and second securement features (116 and 118) are supported berm (153) that form a wall supporting the tube contacting surfaces.

Both the first tube contacting surface (135) and the second tube contacting surface (136) can possess an arcuate transverse cross-section. For example, a region of the first prong (116) can extend horizontally over the base (102) so as to form the first tube contacting surface (135) having an arcuate transverse cross-section, and a region of the second prong (118) can extend horizontally over the base (102) so as to form the second tube contacting surface (136) having an arcuate transverse cross-section.

The first tube contacting surface (135) and the second tube contacting surface (136) can be exposed towards opposing sides of a vertically oriented reference plane disposed between the first location (140) and the second location (142) when the base is horizontally disposed. The region of the first prong (116) and the region of the second prong (118) can extend towards opposing sides of a vertically oriented reference plane disposed between the first location (140) and the second location (142) when the base is horizontally disposed. In certain embodiments, the region of the first prong (116) and the region of the second prong (118) can extend in substantially opposing directions.

In certain embodiments, the arcuate transverse cross-section of the first tube contacting surface (135) and the arcuate transverse cross-section of the second tube contacting surface (136) can each have a radius of curvature equal to from 20% to 49% of the outer diameter of the tube. In certain embodiments, the length of the first tube contacting surface (139) and the length of the second tube contacting surface (141) can each be from 3 mm to 70 mm (e.g., from 10 mm to 30 mm).

As illustrated in FIGS. 1A-1C, in some embodiments, the device can include a third securement feature (120; a clip or channel) projecting from the top surface of the base (106) in the tube-securing region (112) at a third location (144) spaced apart (e.g., by distance 150) and distal to the second location (142). In some embodiments, the third securement feature (120) is a channel (e.g., a linear channel or a serpentine channel) that comprises a third tube contacting surface (137) having an arcuate transverse cross-section. The arcuate transverse cross-section of the third tube contacting surface (137) can have a radius of curvature equal to from 20% to 49% of the outer diameter of the tube. In some embodiments, the third tube contacting surface (137) can have a length (143) of from 3 mm to 70 mm.

As illustrated in FIGS. 1A-1C, the first location (140), the second location (142), and the third location (144) can all be offset or swaled with respect to one another, such that vertically oriented reference planes disposed between the first location and the second location, the first location and the third location, and the second location and the third location when the base is horizontally disposed are not parallel to or coplanar with the central reference plane (114).

As illustrated in FIGS. 1A-1C, in some embodiments, the device can include a tube-inserting region (122). The tube-inserting region (122) can comprise a first arm (124) and a second arm (126). The first arm (124) and the second arm (126) together form an annular member (130) that at least partially defines an aperture (128). The annular member (130) can be a discontinuous annular member that includes an opening (132) along its circumference. The opening (132) has a first dimension deformable to a second dimension. The second dimension can be sized to permit passage of the tube through the opening (132) of the annular member (130) and into the aperture (128). Optionally, the first dimension of the opening (132) can be smaller than the outer diameter of the tube, so as to inhibit passage of the tube through the opening (132) of the annular member (130) when the opening (132) is not deformed. The device further includes a plurality of anchor points (134) positioned symmetrically around the annular member (130).

Referring now to FIGS. 2A-2B, in some embodiments, devices for stabilizing a tube in the body of a subject can include a base (102) comprising a patient contacting surface (104), an opposing top surface (106), a proximal end (108), a distal end (110), a tube-securing region (112), and a vertically oriented central reference plane (114) running from the proximal end of the base (108) to the distal end of the base (110) when the base is horizontally disposed. The devices can further include three securement features (116, 118, and 120) positioned within the tube-securing region (112). The three securement features can be configured to reversibly engage the tube, such that when the tube is engaged by the three securement features, the tube is retained relative to the base.

As illustrated in FIGS. 2A-2B, in some embodiments, the device can include a first securement feature (116; a prong) comprising a first tube contacting surface (135) upwardly projecting from the top surface of the base (106) in the tube-securing region (112) at a first location (140), and a second securement feature (118; a prong) comprising a second tube contacting surface (136) upwardly projecting from the top surface of the base (106) in the tube-securing region at a second location (142) spaced apart and distal to the first location (140).

Both the first tube contacting surface (135) and the second tube contacting surface (136) can possess an arcuate transverse cross-section. For example, a region of the first prong (116) can extend horizontally over the base (102) so as to form the first tube contacting surface (135) having an arcuate transverse cross-section, and a region of the second prong (118) can extend horizontally over the base (102) so as to form the second tube contacting surface (136) having an arcuate transverse cross-section.

The first tube contacting surface (135) and the second tube contacting surface (136) can be exposed towards opposing sides of a vertically oriented reference plane disposed between the first location (140) and the second location (142) when the base is horizontally disposed. The region of the first prong (116) and the region of the second prong (118) can extend towards opposing sides of a vertically oriented reference plane disposed between the first location (140) and the second location (142) when the base is horizontally disposed. In certain embodiments, the region of the first prong (116) and the region of the second prong (118) can extend in substantially opposing directions.

In certain embodiments, the arcuate transverse cross-section of the first tube contacting surface (135) and the arcuate transverse cross-section of the second tube contacting surface (136) can each have a radius of curvature equal to from 20% to 49% of the outer diameter of the tube. In certain embodiments, the length of the first tube contacting surface (139) and the length of the second tube contacting surface (141) can each be from 3 mm to 70 mm (e.g., from 10 mm to 30 mm).

As illustrated in FIGS. 2A-2B, in some embodiments, the device can include a third securement feature (120; a clip or channel) projecting from the top surface of the base (106) in the tube-securing region (112) at a third location (144) spaced apart and distal to the second location (142). In some embodiments, the third securement feature (120) is a channel (e.g., a linear channel or a serpentine channel) that comprises a third tube contacting surface (137) having an arcuate transverse cross-section. The arcuate transverse cross-section of the third tube contacting surface (137) can have a radius of curvature equal to from 20% to 49% of the outer diameter of the tube. In some embodiments, the third tube contacting surface (137) can have a length (143) of from 3 mm to 70 mm.

As illustrated in FIGS. 2A-2B, the first location (140), the second location (142), and the third location (144) can all be positioned along the central reference plane of the device (114).

As illustrated in FIGS. 2A-2B, in some embodiments, the device can include a tube-inserting region (122). The tube-inserting region (122) can comprise a first arm (124) and a second arm (126). The first arm (124) and the second arm (126) together form an annular member (130) that at least partially defines an aperture (128). The annular member (130) can be a discontinuous annular member that includes an opening (132) along its circumference. The opening (132) has a first dimension deformable to a second dimension. The second dimension can be sized to permit passage of the tube through the opening (132) of the annular member (130) and into the aperture (128). Optionally, the first dimension of the opening (132) can be smaller than the outer diameter of the tube, so as to inhibit passage of the tube through the opening (132) of the annular member (130) when the opening (132) is not deformed. The device further includes a plurality of anchor points (134) positioned around the annular member (130). The device further includes a plurality of anchor points (134) positioned elsewhere around the base (e.g., in the tube-securing region (112)).

FIG. 2C shows an perspective view of a device similar to the device illustrated in FIGS. 2A-2B. As illustrated in FIG. 2C, in some embodiments, the device can lack anchor points (134) in the tube securing region (112). FIG. 9 is a schematic drawing of the device illustrated in FIG. 2C, with the dimensions of certain features (in mm) noted.

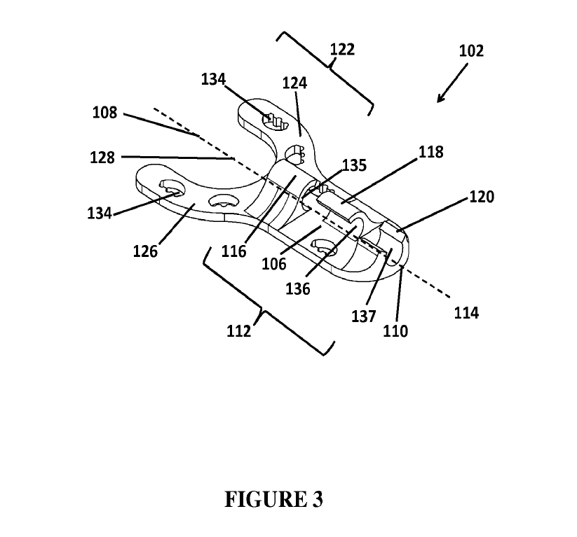

Referring now to FIG. 3, in some embodiments, devices for stabilizing a tube in the body of a subject can include a base (102) comprising a patient contacting surface (104), an opposing top surface (106), a proximal end (108), a distal end (110), a tube-securing region (112), and a vertically oriented central reference plane (114) running from the proximal end of the base (108) to the distal end of the base (110) when the base is horizontally disposed. The devices can further include three securement features (116, 118, and 120) positioned within the tube-securing region (112). The three securement features can be configured to reversibly engage the tube, such that when the tube is engaged by the three securement features, the tube is retained relative to the base.

As illustrated in FIG. 3, in some embodiments, the device can include a first securement feature (116; a prong) comprising a first tube contacting surface (135) upwardly projecting from the top surface of the base (106) in the tube-securing region (112) at a first location, and a second securement feature (118; a prong) comprising a second tube contacting surface (136) upwardly projecting from the top surface of the base (106) in the tube-securing region at a second location spaced apart and distal to the first location.

Both the first tube contacting surface (135) and the second tube contacting surface (136) can possess an arcuate transverse cross-section. For example, a region of the first prong (116) can extend horizontally over the base (102) so as to form the first tube contacting surface (135) having an arcuate transverse cross-section, and a region of the second prong (118) can extend horizontally over the base (102) so as to form the second tube contacting surface (136) having an arcuate transverse cross-section.

The first tube contacting surface (135) and the second tube contacting surface (136) can be exposed towards opposing sides of a vertically oriented reference plane disposed between the first location and the second location when the base is horizontally disposed. The region of the first prong (116) and the region of the second prong (118) can extend towards opposing sides of a vertically oriented reference plane disposed between the first location and the second location when the base is horizontally disposed. In certain embodiments, the region of the first prong (116) and the region of the second prong (118) can extend in substantially opposing directions.

In certain embodiments, the arcuate transverse cross-section of the first tube contacting surface (135) and the arcuate transverse cross-section of the second tube contacting surface (136) can each have a radius of curvature equal to from 20% to 49% of the outer diameter of the tube. In certain embodiments, the length of the first tube contacting surface (139) and the length of the second tube contacting surface (141) can each be from 3 mm to 70 mm (e.g., from 10 mm to 30 mm).

As illustrated in FIG. 3, in some embodiments, the device can include a third securement feature (120; a clip or channel) projecting from the top surface of the base (106) in the tube-securing region (112) at a third location spaced apart and distal to the second location. In some embodiments, the third securement feature (120) is a channel (e.g., a linear channel or a serpentine channel) that comprises a third tube contacting surface (137) having an arcuate transverse cross-section. The arcuate transverse cross-section of the third tube contacting surface (137) can have a radius of curvature equal to from 20% to 49% of the outer diameter of the tube. In some embodiments, the third tube contacting surface (137) can have a length (143) of from 3 mm to 70 mm.

As illustrated in FIG. 3, the first location, the second location, and the third location can all be positioned along the central reference plane of the device (114).

As illustrated in FIG. 3, in some embodiments, the device can include a tube-inserting region (122). The tube-inserting region (122) can comprise a first arm (124) and a second arm (126) that together at least partially define an aperture (128). The device further includes a plurality of anchor points (134) positioned in the tube inserting region (122). The device further includes a plurality of anchor points (134) positioned elsewhere around the base (e.g., in the tube-securing region (112)).

Referring now to FIG. 4A-4B, in some embodiments, devices for stabilizing a tube in the body of a subject can include a base (102) comprising a patient contacting surface (104), an opposing top surface (106), a proximal end (108), a distal end (110), a tube-securing region (112), and a vertically oriented central reference plane (114) running from the proximal end of the base (108) to the distal end of the base (110) when the base is horizontally disposed. The devices can further include a securement feature (116) positioned within the tube-securing region (112). The securement feature can be configured to reversibly engage the tube, such that when the tube is engaged by the securement feature, the tube is retained relative to the base.

As illustrated in FIG. 4A-4B, in some embodiments, the securement feature (116) can be a serpentine channel (154) projecting from the top surface of the base (106) in the tube-securing region (112) that comprises a tube contacting surface (156) having an arcuate transverse cross-section. The arcuate transverse cross-section of the tube contacting surface (156) can have a radius of curvature equal to from 20% to 49% of the outer diameter of the tube. In some embodiments, the tube contacting surface (156) can have a length of from 3 mm to 70 mm.

As illustrated in FIG. 4A-4B, in some embodiments, the device can include a tube-inserting region (122). The tube-inserting region (122) can comprise a first arm (124) and a second arm (126). The first arm (124) and the second arm (126) together form an annular member (130) that at least partially defines an aperture (128). The annular member (130) can be a discontinuous annular member that includes an opening (132) along its circumference. The opening (132) has a first dimension deformable to a second dimension. The second dimension can be sized to permit passage of the tube through the opening (132) of the annular member (130) and into the aperture (128). Optionally, the first dimension of the opening (132) can be smaller than the outer diameter of the tube, so as to inhibit passage of the tube through the opening (132) of the annular member (130) when the opening (132) is not deformed. The device further includes a plurality of anchor points (134) positioned around the annular member (130).

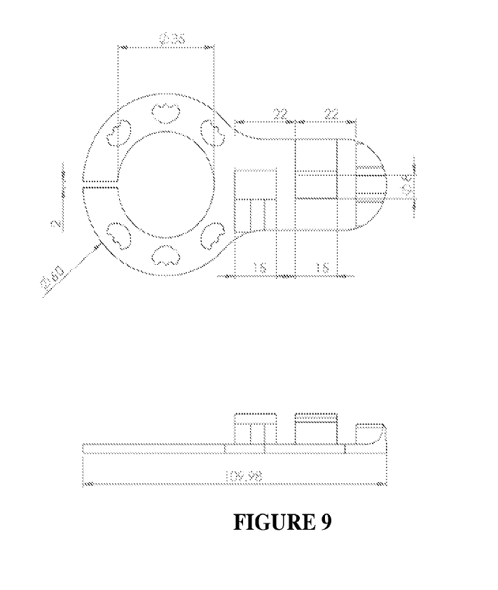

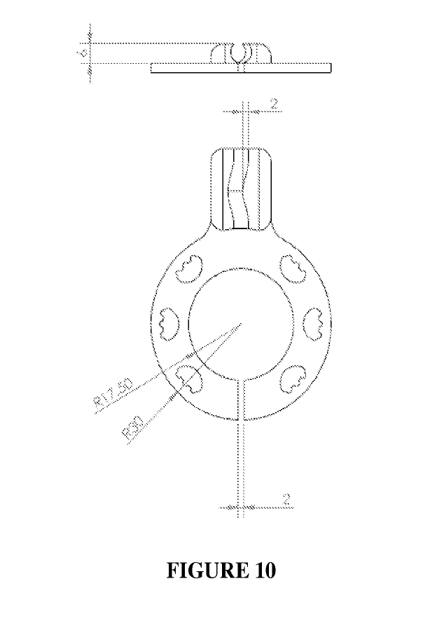

FIG. 10 is a schematic drawing of the device illustrated in FIGS. 4A-4B, with the dimensions of certain features (in mm) noted.

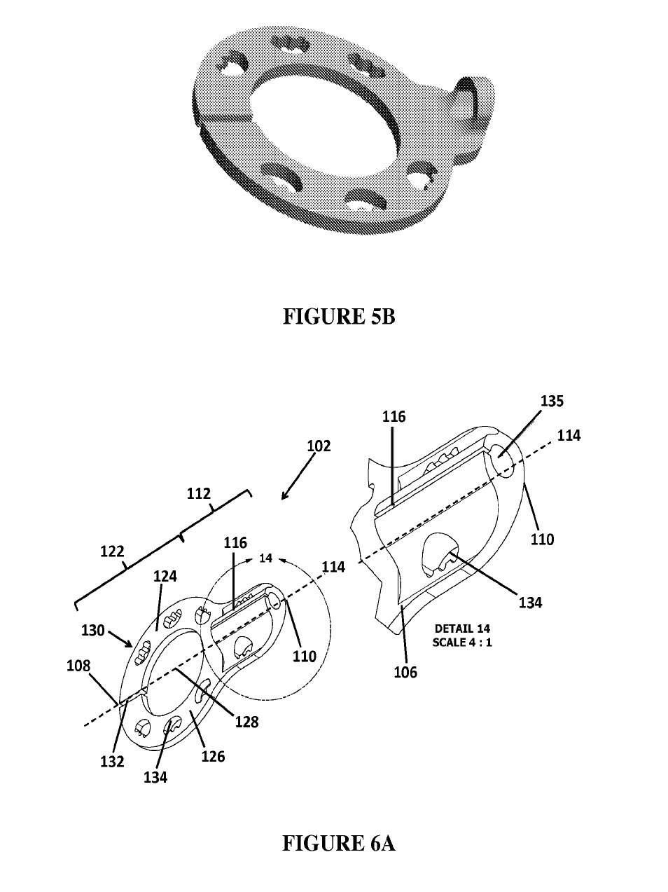

Referring now to FIG. 5A-5B, in some embodiments, devices for stabilizing a tube in the body of a subject can include a base (102) comprising a patient contacting surface (104), an opposing top surface (106), a proximal end (108), a distal end (110), a tube-securing region (112), and a vertically oriented central reference plane (114) running from the proximal end of the base (108) to the distal end of the base (110) when the base is horizontally disposed. The devices can further include a securement feature (116) positioned within the tube-securing region (112). The securement feature can be configured to reversibly engage the tube, such that when the tube is engaged by the securement feature, the tube is retained relative to the base.

As illustrated in FIG. 5A-5B, in some embodiments, the securement feature (116) can be a clip projecting from the top surface of the base (106) in the tube-securing region (112) that comprises a tube contacting surface (135) having an arcuate (e.g., circular or ellipsoid) transverse cross-section. The arcuate transverse cross-section of the tube contacting surface (135) can have a radius of curvature equal to from 20% to 49% of the outer diameter of the tube. In some embodiments, the tube contacting surface (135) can have a length of from 3 mm to 70 mm.

As illustrated in FIG. 5A-5B, in some embodiments, the device can include a tube-inserting region (122). The tube-inserting region (122) can comprise a first arm (124) and a second arm (126). The first arm (124) and the second arm (126) together form an annular member (130) that at least partially defines an aperture (128). The annular member (130) can be a discontinuous annular member that includes an opening (132) along its circumference. The opening (132) has a first dimension deformable to a second dimension. The second dimension can be sized to permit passage of the tube through the opening (132) of the annular member (130) and into the aperture (128). Optionally, the first dimension of the opening (132) can be smaller than the outer diameter of the tube, so as to inhibit passage of the tube through the opening (132) of the annular member (130) when the opening (132) is not deformed. The device further includes a plurality of anchor points (134) positioned around the annular member (130).

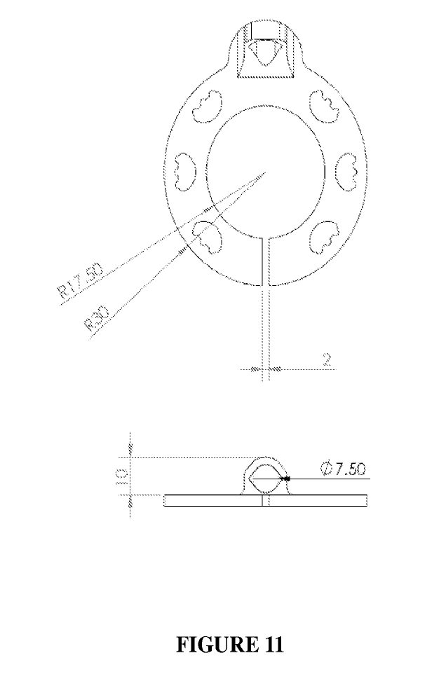

FIG. 11 is a schematic drawing of the device illustrated in FIGS. 5A-5B, with the dimensions of certain features (in mm) noted

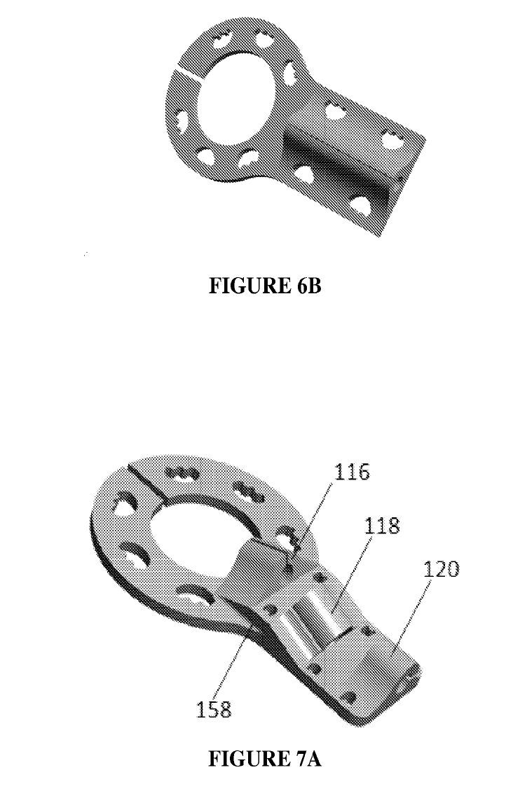

Referring now to FIG. 6A, in some embodiments, devices for stabilizing a tube in the body of a subject can include a base (102) comprising a patient contacting surface (104), an opposing top surface (106), a proximal end (108), a distal end (110), a tube-securing region (112), and a vertically oriented central reference plane (114) running from the proximal end of the base (108) to the distal end of the base (110) when the base is horizontally disposed. The devices can further include a securement feature (116) positioned within the tube-securing region (112). The securement feature can be configured to reversibly engage the tube, such that when the tube is engaged by the securement feature, the tube is retained relative to the base.

As illustrated in FIG. 6A, in some embodiments, the securement feature (116) can be a linear channel (116) projecting from the top surface of the base (106) in the tube-securing region (112) that comprises a tube contacting surface (135) having an arcuate transverse cross-section. The arcuate transverse cross-section of the tube contacting surface (135) can have a radius of curvature equal to from 20% to 49% of the outer diameter of the tube. In some embodiments, the tube contacting surface (135) can have a length of from 3 mm to 70 mm.

As illustrated in FIG. 6A, in some embodiments, the device can include a tube-inserting region (122). The tube-inserting region (122) can comprise a first arm (124) and a second arm (126). The first arm (124) and the second arm (126) together form an annular member (130) that at least partially defines an aperture (128). The annular member (130) can be a discontinuous annular member that includes an opening (132) along its circumference. The opening (132) has a first dimension deformable to a second dimension. The second dimension can be sized to permit passage of the tube through the opening (132) of the annular member (130) and into the aperture (128). Optionally, the first dimension of the opening (132) can be smaller than the outer diameter of the tube, so as to inhibit passage of the tube through the opening (132) of the annular member (130) when the opening (132) is not deformed. The device further includes a plurality of anchor points (134) positioned symmetrically around the annular member (130). The device further includes a plurality of anchor points (134) positioned elsewhere around the base (e.g., in the tube-securing region (112)).

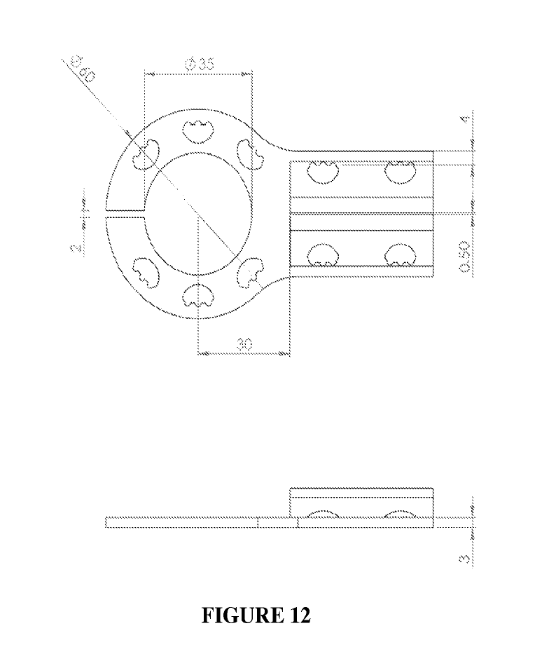

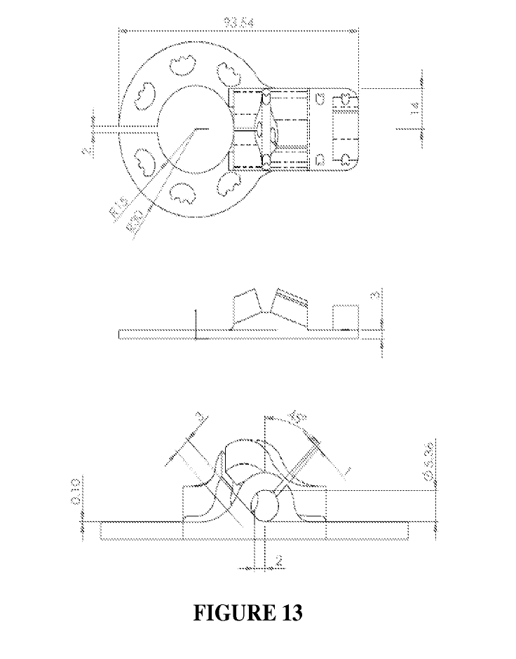

FIG. 6B shows an perspective view of a device similar to the device illustrated in FIG. 6A. As illustrated in FIG. 6B, in some embodiments, the device can lack anchor points (134) in the tube securing region (112). FIG. 12 is a schematic drawing of the device illustrated in FIG. 6B, with the dimensions of certain features (in mm) noted.

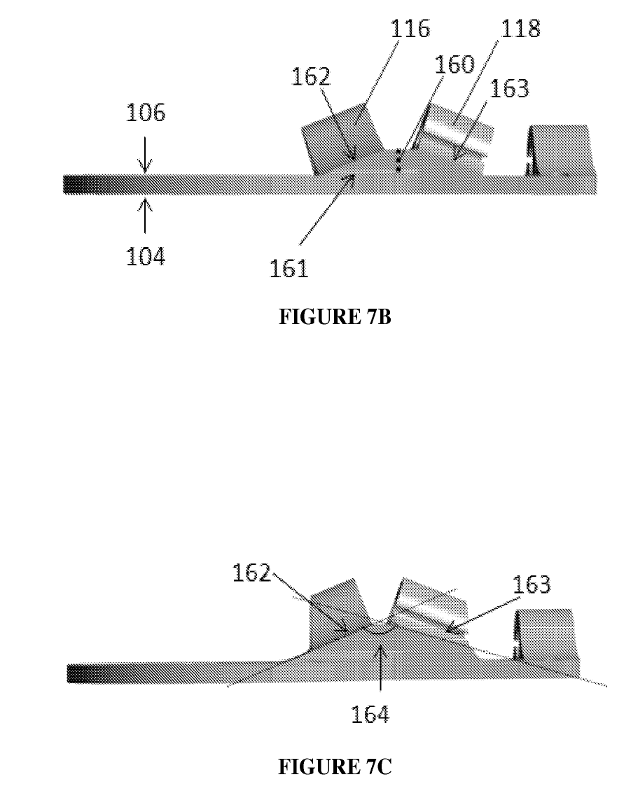

Referring now to FIGS. 7A-7C, in some embodiments, one or more of the securement features can be positioned on a platform (158). The platform can be of any suitable design, and can be configured to position one or more of the securement features above the plane of the top surface of the base. Referring to FIG. 7A, in some embodiments, the first securement feature (116) and the second securement feature (116 and 118) can be positioned on a platform (158).

Referring now to the longitudinal cross-section shown in FIG. 7B, the platform (158) can be of varying heights. For example, the greatest height of the platform (160) can be 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, or 30 mm. The greatest height of the platform can range between any of the values above. For example, the greatest height of the platform can be from 1 mm to 30 mm (e.g., from 1 mm to 15 mm, from 1 mm to 10 mm, or from 1 mm to 5 mm). In some embodiments, the greatest height of the platform can be at least 50% of the thickness of the base (102) (i.e., the distance between the patient contacting surface of the base (104) and the top surface of the base (106)).

In some embodiments, the longitudinal cross-section of the platform can be defined three or more surfaces. For example, the platform can include a bottom surface (161), a first securement feature supporting surface (162), and a second securement feature supporting surface (163). The bottom surface of the platform (161) can be in contact with the top surface of the base (106), and the first securement feature supporting surface (162) and the second securement feature supporting surface (163) can be exposed such that each surface can support a securement feature, as illustrated in FIGS. 7B-7C.

In some cases, the first securement feature supporting surface (162) and/or the second securement feature supporting surface (163) can be tilted at an angle with respect to the base, such that the plane of the first securement feature supporting surface (162) and/or the plane of the second securement feature supporting surface (163) is not parallel to the plane of the top surface of the base (106). In some embodiments, both the first securement feature supporting surface (162) and the second securement feature supporting surface (163) can be tilted at an angle with respect to the base. In some cases, the plane of the first securement feature supporting surface (162) and the plane of the second securement feature supporting surface (163) intersect at an angle (164) that is 179 degrees or less (e.g., from 45-179 degrees, 90-179 degrees, or 120-160 degrees). In this way, a vertical swale can be incorporated in a device and/or a device can be configured to more readily engage a tube inserted at a shallow angle within a subject.

Devices for stabilizing a tube in the in the body of a subject can be of a single piece construction and can be formed by use of a single mold and of a uniform material. As such, no assembly of pieces is required during use of the device. This can simplify manufacturing of the device, leading to lower costs, as well as simplify utilization of the device. In general, the device 10 can be formed of a moldable biocompatible, sterilizable polymeric material, such as a silicone elastomer, a polyurethane, or another suitable polymer as is generally known in the art. Optionally, the device is formed of a biocompatible material. For example, the device is optionally formed from a biocompatible silicone elastomer. Optionally, the silicone elastomer has a Shore A hardness of from 30 to 60, as measured by DIN 53505. For example, the Shore A hardness is optionally 30, 35, 40, 45, 50, 55, 60, or hardness values in between these values. Optionally, the silicone elastomer has a tensile strength of from 6 to 12 N/mm² as measured by DIN 53504 S 1. For example, the tensile strength is optionally 6, 7, 8, 9, 10, 11, 12 N/mm² or values in between these values. Optionally, the silicone elastomer has a tear strength of from 20 N/mm to 70 N/mm, as measured by ASTM D624 B. For example, the tear strength is optionally 20, 25, 30, 35, 40, 45, 50, 55, 60, 65, 70, or values between these values. The device can be formed according to standard methodology, for instance according to an injection molding process as is known.

Method of Using the Stabilizers.

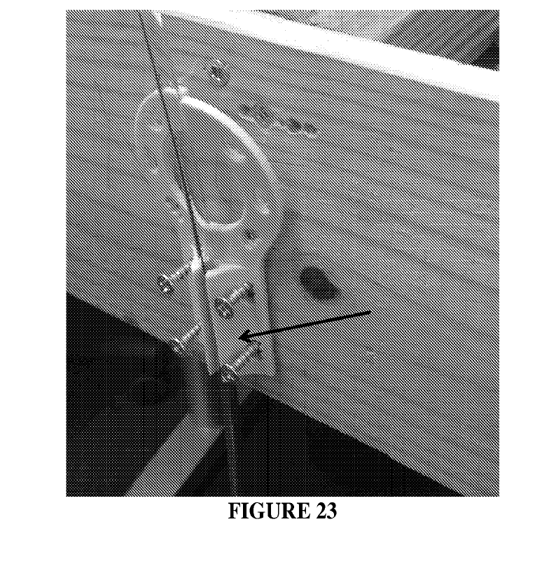

The disclosed stabilizers can be used in a variety of situations, including chest or abdominal tube placements, or catheter placements, such as a Hickman catheter placement. The stabilizers can be used by attaching the stabilizer to the patient, via sutures. One, two, three, four, five, six, or more sutures may be used. The stabilizer can also be secured by other means (e.g., tape and/or an adhesive). After placement, the tube to be secured, such as a chest tube, abdominal tube, or catheter, is secured on the stabilizer through insertion of the tube into or within the one or more securement features of the device. FIG. 23 is a photograph showing a tube secured in a device for stabilizing a tube in the body of a subject. Note that the tube is engaged by the securement feature in the device such that it is retained relative to the base.

Also disclosed are the components to be used to prepare the disclosed devices as well as the devices themselves to be used within the methods disclosed herein. These and other materials are disclosed herein, and it is understood that when combinations, subsets, interactions, groups, etc. of the components making up a device are disclosed that while specific reference of each various individual and collective combination and permutation of these components may not be explicitly disclosed, each is specifically contemplated and described herein. Thus, if a device formed from A, B, and C is disclosed as well as a components D, E, and F and an example of a combination, A-D is disclosed, then even if each is not individually recited each is individually and collectively contemplated meaning combinations, A-E, A-F, B-D, BE, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application, including particularly the components of the devices described herein.

It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data are provided in a number of different formats, and that this data, represents endpoints and starting points, and ranges for any combination of the data points. For example, if a particular datum point “10” and a particular datum point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, yet the absence of a particular advantage shall not be construed to necessarily mean that such an embodiment is outside the scope of the present invention.

### Embodiments

The following is a non-exclusive list of exemplary embodiments in accordance with the present disclosure:

1. A device for stabilizing a tube in the body of a subject, the device comprising:

(a) a base configured to be secured to a patient comprising a patient contacting surface, an opposing top surface, a proximal end, a distal end, a tube-securing region, and a central reference plane; and

(b) one or more securement features positioned within the tube-securing region, wherein the one or more securement features are configured to reversibly engage the tube such that when the tube is engaged by the one or more securement features, the tube is retained relative to the base.

2. The device of embodiment 1, wherein the one or more securement features are configured such that when the tube is engaged by the one or more securement features, the one or more securement features apply at least 2 N of securement force to the tube.
3. The device of embodiment 1 or 2, wherein the one or more securement features are configured such that when the tube is engaged by the one or more securement features, the one or more securement features apply from 2 to 20 N of securement force to the tube.
4. The device of any of embodiments 1-3, wherein the base further comprises a tube inserting region.
5. The device of any of embodiments 1-4, wherein the base further comprises a plurality of anchor points.
6. The device of embodiment 5, wherein an anchor point is positioned within the tube-inserting region.
7. The device of embodiment 5 or 6, wherein the plurality of anchor points each individually comprise an eyelet or hook.
8. The device of any of embodiments 5-7, wherein the plurality of anchor points are positioned symmetrically around the base.
9. The device of any of embodiments 4-8, wherein the tube-inserting region comprises a first arm and a second arm that together at least partially define an aperture sized to permit passage of the tube through the aperture from a point above the top surface of the device to a point below the patient contacting surface of the device.
10. The device of embodiment 9, wherein first arm and a second arm together form an annular member that at least partially defines the aperture.
11. The device of embodiment 10, wherein the annular member is a continuous annular member.
12. The device of claim 10, wherein the annular member is a discontinuous annular member.
13. The device of embodiment 12, wherein the discontinuous annular member comprises an opening having a first dimension deformable to a second dimension.
14. The device of embodiment 13, wherein the second dimension is sized to permit passage of the tube through the opening of the annular member and into the aperture.
15. The device of any of embodiments 10-14, wherein the annular member comprises a plurality of anchor points.
16. The device of embodiment 15, wherein the plurality of anchor points each individually comprise eyelets, hooks, or loops.
17. The device of embodiment 15 or 16, wherein the plurality of anchor points are positioned symmetrically around the annular member.
18. The device of any of embodiments 1-17, wherein the one or more securement features are individually chosen from a prong, a clip, a channel, or combinations thereof.
19. The device of embodiment 18, wherein the one or more securement features comprise a channel comprising a tube contacting surface having an arcuate transverse cross-section.
20. The device of embodiment 19, wherein the arcuate transverse cross-section of the tube contacting surface has a radius of curvature equal to from 20% to 49% of the outer diameter of the tube.
21. The device of embodiment 19 or 20, wherein the tube contacting surface has a length of from 3 mm to 70 mm.
22. The device of any of embodiments 17-21, wherein the channel comprises a serpentine channel.
23. The device of any of embodiments 1-22, wherein the device comprises two or more securement features positioned within the tube-securing region.
24. The device of any of embodiments 1-23, wherein the one or more securement features comprise

(a) a first securement feature positioned within the tube-securing region at a first location; and

(b) a second securement feature positioned within the tube-securing region at a second location spaced apart and distal to the first location.

25. The device of embodiments 24, wherein the first location, the second location, or both the first location and the second location are positioned along the central reference plane.
26. The device of embodiment 25, wherein one of the first location and the second location is positioned along the central reference plane.
27. The device of embodiment 24, wherein the first location and the second location are positioned on opposite sides of the central reference plane.
28. The device of any of embodiments 24-27, wherein the first location and the second location are offset from one another relative to the central reference plane by an offset distance, such that a vertically oriented reference plane disposed between the first location and the second location when the base is horizontally disposed is not parallel to or coplanar with the central reference plane.
29. The device of embodiment 28, wherein the offset distance is from 1 mm to 15 mm.
30. The device of any of embodiments 24-29, further comprising a third securement feature positioned within the tube-securing region at a third location spaced apart and distal to the second location.
31. The device of embodiment 30, wherein the first location, the second location, and the third location are all offset from one another relative to the central reference plane, such that vertically oriented reference planes disposed between the first location and the second location, the first location and the third location, and the second location and the third location when the base is horizontally disposed are not parallel to or coplanar with the central reference plane.
32. The device of embodiment 30, wherein the second location is offset from the first location and the third location relative to the central reference plane, such that vertically oriented reference planes disposed between the first location and the second location and the second location and the third location when the base is horizontally disposed are not parallel to or coplanar with the central reference plane, but a vertically oriented reference plane disposed between the first location the third location is parallel to or coplanar with the central reference plane.
The device of any of embodiments 1-32, wherein the one or more securement features comprise

a first prong comprising a first tube contacting surface upwardly projecting from the top surface of the base in the tube-securing region at a first location, and

a second prong comprising a second tube contacting surface upwardly projecting from the top surface of the base in the tube-securing region at a second location spaced apart and distal to the first location.

34. The device of embodiment 33, wherein the first tube contacting surface and the second tube contacting surface are exposed towards opposing sides of a vertically oriented reference plane disposed between the first location and the second location when the base is horizontally disposed.
35. The device of embodiment 32 or 33, wherein the first tube contacting surface and the second tube contacting surface each possess an arcuate transverse cross-section.
36. The device of any of embodiments 33-35, wherein a region of the first prong extends horizontally over the base so as to form the first tube contacting surface having an arcuate transverse cross-section,

wherein a region of the second prong extends horizontally over the base so as to form the second tube contacting surface having an arcuate transverse cross-section, and

wherein the region of the first prong and the region of the second prong extend towards opposing sides of a vertically oriented reference plane disposed between the first location and the second location when the base is horizontally disposed.

37. The device of embodiment 36, wherein the region of the first prong and the region of the second prong extend in substantially opposing directions.
38. The device of any of embodiments 35-37, wherein the arcuate transverse cross-section of the first tube contacting surface and the arcuate transverse cross-section of the second tube contacting surface each have a radius of curvature equal to from 20% to 49% of the outer diameter of the tube.
39. The device of any of embodiments 33-38, wherein the first tube contacting surface and the second tube contacting surface each have a length of from 3 mm to 70 mm.
40. The device of any of embodiments 33-39, wherein the first tube contacting surface and the second tube contacting surface each have a length of from 10 mm to 30 mm.
41. The device of any of embodiments 33-40, further comprising a clip or channel projecting from the top surface of the base in the tube-securing region at a third location spaced apart and distal to the second location.
42. The device of embodiment 41, wherein the channel comprises a third tube contacting surface having an arcuate transverse cross-section.
43. The device of embodiment 42, wherein the arcuate transverse cross-section of the third tube contacting surface has a radius of curvature equal to from 20% to 49% of the outer diameter of the tube.
44. The device of embodiment 42 or 43, wherein the third tube contacting surface has a length of from 3 mm to 70 mm.
45. The device of any of claims 41-44, wherein the channel comprises a serpentine channel.
46. The device of any of embodiments 1-45, wherein the device is formed of a biocompatible material.
47. The device of embodiment 46, wherein the device is formed from a biocompatible silicone elastomer.
48. The device of embodiment 47, wherein the silicone elastomer has a Shore A hardness of from 30 to 60, as measured by DIN 53505.
49. The device of embodiment 47 or 48, wherein the silicone elastomer has a tensile strength of from 6 to 12 N/mm2 as measured by DIN 53504 S 1.
50. The device of any of embodiments 47-49, wherein the silicone elastomer has a tear strength of from 20 N/mm to 70 N/mm, as measured by ASTM D62.

### EXAMPLES

Experiments were performed to identify the relationship between aspects of device design, characteristics of the tube to be stabilized, and the ability of the device to retain the tube. Variables evaluated included the properties of the substrate material used to fabricate the device, the design of the one or more securement features in the device, and the nature of the tube being secured by the device.

Materials and Methods

Manufacture of Devices

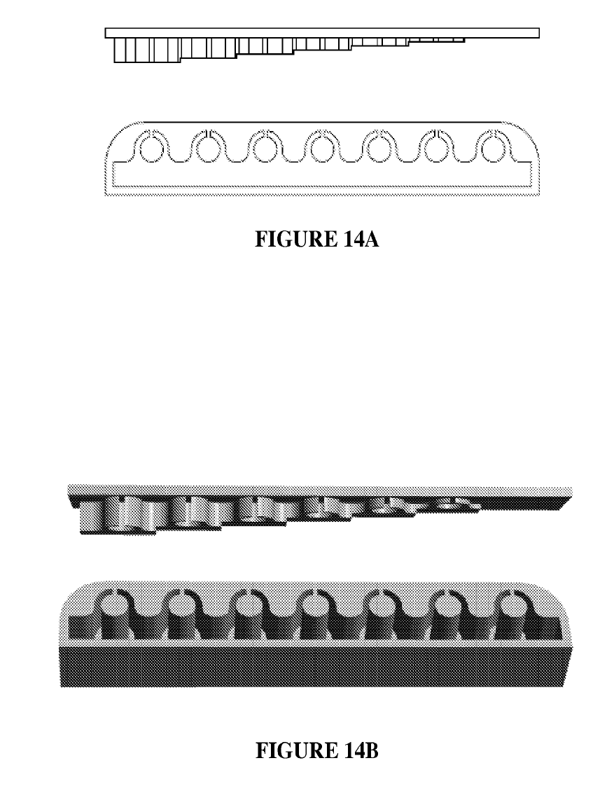

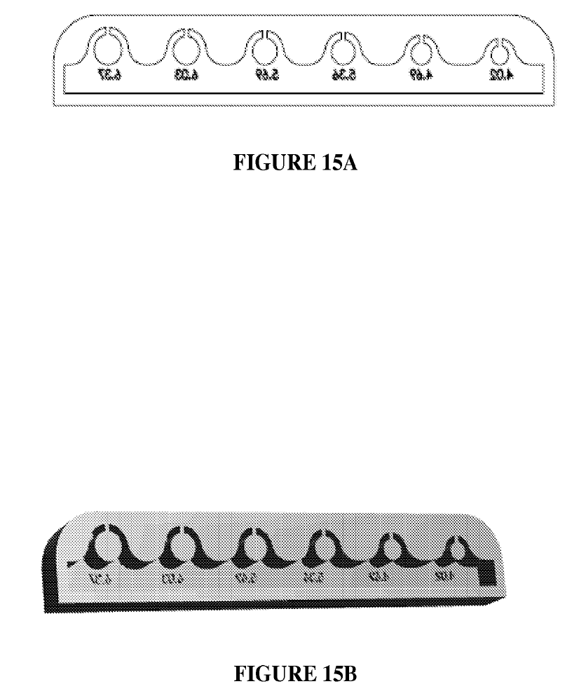

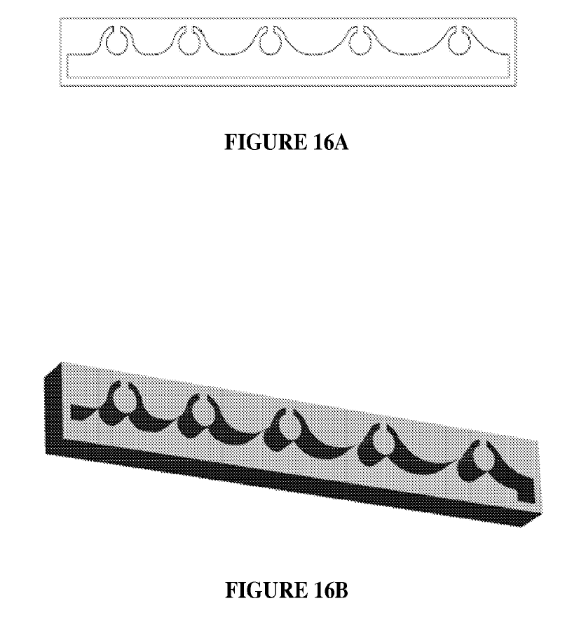

Molds for shaping different securement features were produced using a rapid prototyping machine. FIGS. 14A-14B, 15A-15B, and 16A-16B show molds used to prepare jigs containing a variety of securement features to be evaluated, including securement features having tube contacting surfaces of varying lengths (FIGS. 14A-B), securement features having arcuate tube contacting surfaces with varying radii of curvature (FIGS. 15A-B), and securement features with varying berm (FIGS. 16A-B).

After production of each mold, silicone positives were made from each mold using three different silicones harnesses: Shore A 40 silicone, Shore A 50 silicone, and Shore A 60 silicone.

Each mold was cast with a silicone elastomer, mixed and cast using manufacturer's instructions. After casting and setting each silicone positive was removed and cleaned, and readied for testing.

Measurement of Securement Force

Each silicone positive was placed in an edge-controlled vice so that the linear axis (in line with the linear axis of the tube) securement feature was oriented perpendicular to gravity (the floor).

One PVC elastomer tube and one silicone elastomer tube were cut to a fixed length, and marked to aid in visualizing any movement of the tube relative to the base. Each tube was a 20FR tube having a diameter at the testing position of 6.7 mm. Each tube was then “nicked” with an approximately 45 degree cut near the bottom (floor side, when tube was in position with the securement feature) so that a small “bucket” with a wire “handle” could be hung on the tube with the bucket portion below the end of the “floor” end of the tube.

For each experiment, the tube was placed into the securement feature to be tested, aligned so the mark on the tube was just at the top (ceiling side) of the securement feature, and then the bucket was hung on the tube by placing the wire handle in the 45 degree cut in the tube. Then, water (and in certain cases metal weights) were added to the bucket until when the tube would just begin to move down, through the securement feature.

After this movement, the bucket was removed and the bucket handle was placed onto a hanging scale (SR-1 by “American Weight Scales,” 1000 g max increasing by 1 g increments, Tolerance: ±2 g at 1 kg). The weight of the bucket was recorded. Each securement feature was tested in triplicate.

Statistics and Curves

Data was collected and plotted using Prism 6 for Mac OS X. Data was plotted using three separate Y values, plotted with error bars, using standard mean and standard deviation analysis. In certain cases a two-way ANOVA analysis was done. Fitting of curves is discussed for each particular experiment. For linear curve fitting, the curve was forced through a 0,0 X, Y point, as there would be no force exerted if there was no securement feature. As forces and lengths involved are relatively low, the linear curve fitting method was assumed to provide a reasonable estimate. If the actual data was collected to “saturation,” it might be possible to more accurately model the system using a non-linear fit.

Evaluation of the Relationship Between Length of the Tube Contacting Surface and Securement Force

The effect of the length of contact between the securement feature (i.e., the length of the tube contacting surface) and the tube was tested. Securement features having a diameter of 90% the width of the outer tube diameter were tested. The securement features had tube contacting surfaces of 5, 6, 7, 8, 9, 10, and 11 mm. The securement feature had a berm of 4 mm. A 20FR tube was used for initial investigation.

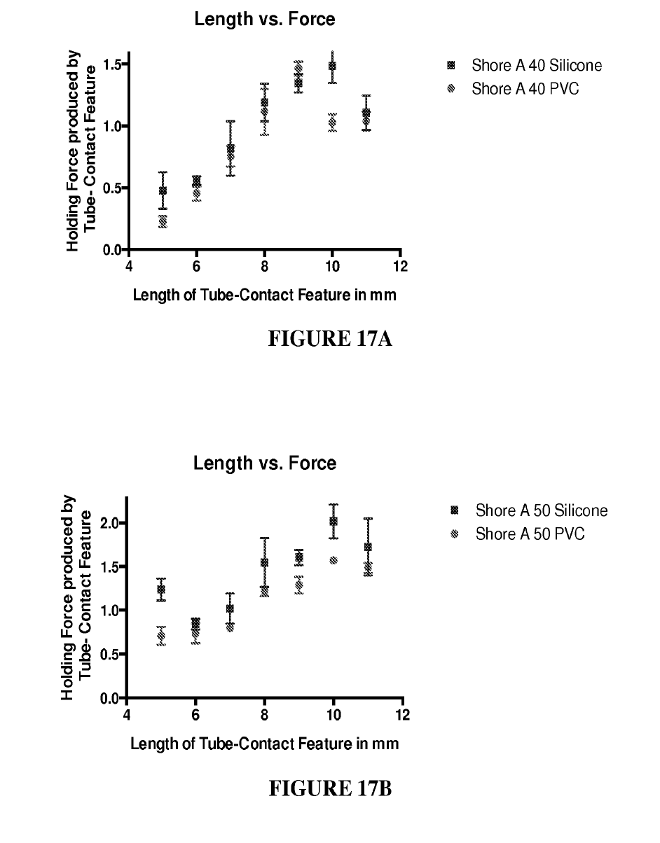

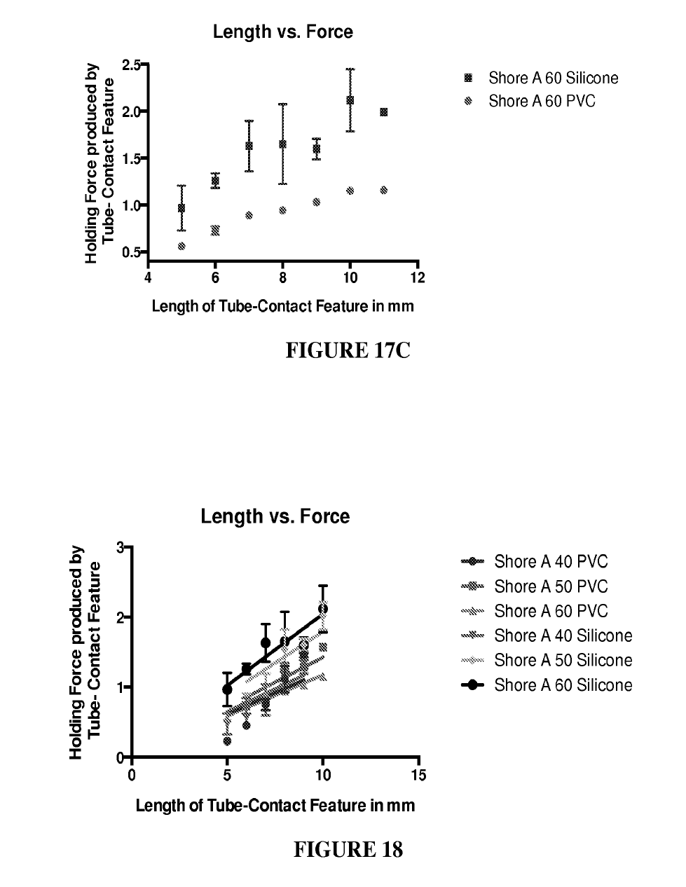

FIGS. 17A-17C shows the results of these experiments for both the silicone tube and the PVC tube (FIG. 17A for Shore A 40, FIG. 17B for Shore A 50, and FIG. 17C for Shore A 60). As shown in FIG. 18, for all six material types, increasing the length of the tube contacting surface increased the amount of securement force generated by the securement feature.

As shown in FIGS. 17A-17C, at the upper lengths tested (10 and 11 mm,), a change in the relationship between force and length was observed. At these higher lengths, the effect of length on force appeared to decrease. The magnitude of the observed decrease was greater in the case of securement featured fabricated from softer materials (i.e., the decrease was greater in the securement features prepared from a Shore A 40 material as compared to a Shore A 50 material, while the decrease was greater in the securement features prepared from a Shore A 50 material as compared to a Shore A 60 material).

Difference Between Hardness and Material

Table 1 shows the raw data generated for the experiments looking at the relationship between force and length. A one-way ANOVA analysis was performed across the averages of each data set (e.g., Shore A 40 silicone) shown in Table 1, looking at both differentiation between the sets of data. This analysis showed that there was statistically significant difference between the three different hardness values (40, 50, 60) for the PVC tube alone, with a p value of 0.0002. Likewise the data also showed that there was a statistical difference between the three different hardness values for the silicone tube, with a p value of 0.0002.

As expected, the one-way ANOVA showed that when comparing all of the three hardness data sets for silicone and PVC, they were significantly different, with a p value of less than 0.0001. The ANOVA analysis suggested that there is a statistically significant difference between the securement force exerted by a securement feature and the material used to fabricate the securement feature.

TABLE 1
Raw force data generated during evaluation of the relationship between
length of the tube contacting surface and the securement force

| Length | 40 | | | 50 | | | 60 | | |
|---|---|---|---|---|---|---|---|---|---|
| mm | Data Set 1 | Data Set 2 | Data Set 3 | Data Set 1 | Data Set 2 | Data Set 3 | Data Set 1 | Data Set 2 | Data Set 3 |

Silicone

| | | | | | | | | | |
|---|---|---|---|---|---|---|---|---|---|
| 5 | 0.3822 | 0.4018 | 0.6468 | 1.1564 | 1.176 | 1.3818 | 0.7546 | 0.9212 | 1.225 |
| 6 | 0.5194 | 0.5684 | 0.588 | 0.784 | 0.9114 | 0.833 | 1.2936 | 1.3132 | 1.1662 |
| 7 | 0.5978 | 1.0388 | 0.8134 | 0.8232 | 1.0878 | 1.1466 | 1.3328 | 1.7052 | 1.8522 |
| 8 | 1.2348 | 1.3132 | 1.0192 | 1.2838 | 1.8424 | 1.5092 | 1.3328 | 2.1364 | 1.4798 |
| 9 | 1.2642 | 1.4112 | 1.3622 | 1.5582 | 1.5484 | 1.7052 | 1.6758 | 1.6464 | 1.47 |
| 10 | 1.617 | 1.4896 | 1.3426 | 1.8032 | 2.1854 | 2.058 | 1.7346 | 2.352 | 2.2638 |
| 11 | 1.2544 | 0.98 | 1.078 | 1.4014 | 2.058 | 1.7052 | 1.9894 | | |

PVC

| | | | | | | | | | |
|---|---|---|---|---|---|---|---|---|---|
| 5 | 0.2058 | 0.196 | 0.2744 | 0.8232 | 0.637 | 0.6566 | 0.5684 | 0.5782 | 0.5292 |
| 6 | 0.4312 | 0.4116 | 0.5194 | 0.6076 | 0.8232 | 0.7742 | 0.7448 | 0.7644 | 0.6762 |
| 7 | 0.8036 | 0.6566 | 0.7938 | 0.7644 | 0.8428 | 0.784 | 0.9212 | 0.882 | 0.8624 |
| 8 | 0.9114 | 1.1564 | 1.274 | 1.1662 | 1.2054 | 1.274 | 0.9702 | 0.9506 | 0.9016 |
| 9 | 1.5092 | 1.4014 | 1.4798 | 1.176 | 1.3524 | 1.3328 | 1.0388 | 1.0192 | 1.029 |
| 10 | 0.9604 | 1.0192 | 1.0976 | 1.568 | 1.5778 | 1.5582 | 1.1368 | 1.1662 | 1.1466 |
| 11 | 1.0878 | 1.078 | 0.9506 | 1.421 | 1.5092 | 1.5288 | 1.127 | 1.176 | 1.1662 |

Analysis of Force/Length Relationship

FIG. 18 shows the plot of all six materials tested (with outliers removed) and fit to a linear curve. The statistics and calculated values for each of these curves is shown in Table 2, including the calculated slope for each material and for each data set.

TABLE 2
Calculated Slopes for Data Sets for Shore A 40, 50, and 60 material

| | Shore A 40 | Shore A 50 | Shore A60 | Shore A 40 | Shore A 50 | Shore A 60 |
|---|---|---|---|---|---|---|
| Best-fit values | PVC | PVC | PVC | Silicone | Silicone | Silicone |
| Slope | 0.1223 ± 0.01086 | 0.1423 ± 0.004620 | 0.1174 ± 0.001332 | 0.1297 ± 0.007628 | 0.1792 ± 0.007749 | 0.2043 ± 0.008167 |
| 95% Confidence | | | | | | |
| Intervals | | | | | | |
| Slope | 0.09903 to | 0.1324 to | 0.1146 to | 0.1134 to | 0.1626 to | 0.1871 to |
| | 0.1456 | 0.1523 | 0.1202 | 0.1461 | 0.1958 | 0.2216 |
| Goodness of Fit | | | | | | |
| Sy.x | 0.3004 | 0.1454 | 0.04347 | 0.211 | 0.2438 | 0.2665 |
| Is slope significantly | | | | | | |
| non-zero? | | | | | | |
| T | 11.26 | 30.81 | 88.12 | 17 | 23.13 | 25.02 |
| DF | 14 | 14 | 17 | 14 | 14 | 17 |
| P value | <0.0001 | <0.0001 | <0.0001 | <0.0001 | <0.0001 | <0.0001 |
| Deviation | Significant | Significant | Significant | Significant | Significant | Significant |
| from zero? | | | | | | |
| Data | | | | | | |
| Number of X | 5 | 5 | 6 | 5 | 5 | 6 |
| values | | | | | | |
| Maximum | 3 | 3 | 3 | 3 | 3 | 3 |
| number of Y | | | | | | |
| replicates | | | | | | |
| Total number | 15 | 15 | 18 | 15 | 15 | 18 |
| of values | | | | | | |
| Number of | 6 | 6 | 3 | 6 | 6 | 3 |
| missing values | Y = 0.1223 * X | Y = 0.1423 * X | Y = 0.1174 * X | Y = 0.1297 * X | Y = 0.1792 * X | Y = 0.2043 * X |
| Equation | −0.0 | −0.0 | −0.0 | −0.0 | −0.0 | −0.0 |

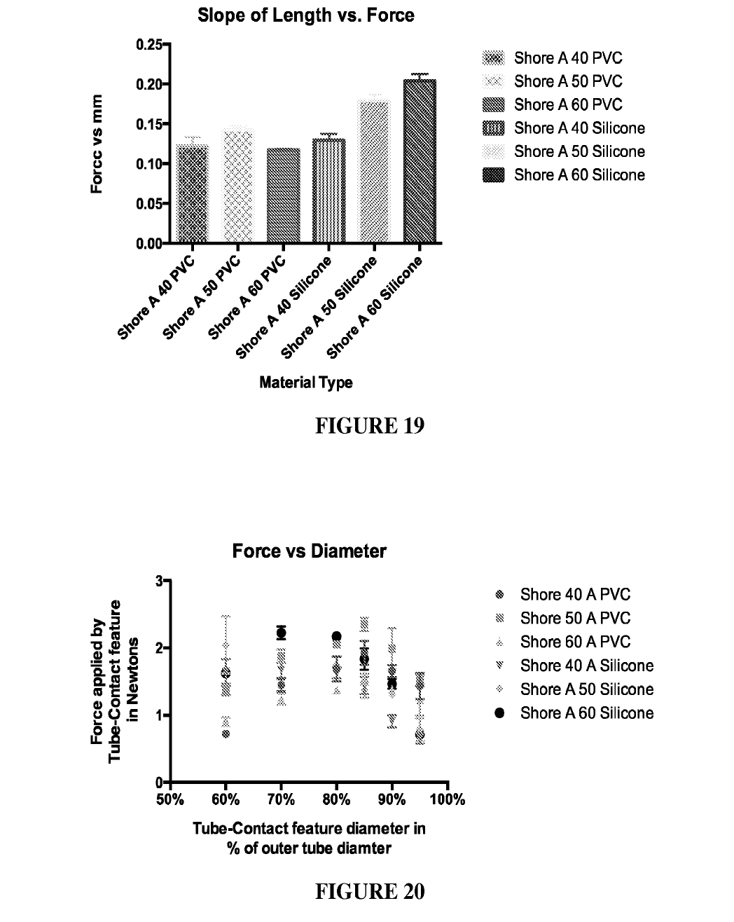

FIG. 19 shows a bar graph of the calculated slopes in Table 2. Based on an initial analysis of the error bars, it appears that the slopes for each best-fit line are significantly different. The data in FIG. 19 and in Table 2 indicate that the securement feature force is greater for silicone tubes vs. PVC tubes. The data also shows that in the case of silicone tubes, securement features formed from harder materials exert increased securement force on the tube.

Taken as a whole, the data suggest that that there is a relationship between the length of the tube contacting surface and the amount of securement force the securement feature is able to exert on a tube placed in contact with the feature. As the length of the tube contacting surface increases, the amount of securement force applied by the securement feature increases. This increase was at a first approximation a linear in the range of the data tested.

Furthermore, the data suggest that there is a relationship between the material used for to form a securement feature and the securement force applied by the feature. Securement featured were formed from three silicones of varying hardness: Shore A 40, Shore A 50, and Shore A60 silicone. As the hardness increased, the securement force exerted on the tube increased. Furthermore, the increase in securement force as a function of length of the tube contacting surface also increased. As a consequence, the additive securement force for each mm increase in the length of the tube contacting surface increased as the hardness of the material increased.

Evaluation of the Relationship Between Diameter of the Tube Contacting Surface and Securement Force

The effect of the diameter of the arcuate tube contacting surface and the tube was tested. The securement features were tested at a length of 8 mm and a berm of 4 mm. The diameter of the lumen formed by the tube contacting surface was tested at 60% (4.02 mm for 20FR), 70% (4.69 mm for 20FR), 80% (5.36 mm for 20FR), 85% (5.69 mm for 20FR), 90% (6.03 mm for 20FR), and 95% (6.37 mm for 20FR) of the outer diameter of the tube. The radii of curvature of the same inner lumens were 30%, 35%, 40%, 42.5%, 45%, and 47.5% of the outer diameter of the tube (20FR). FIG. 20 shows a plot of the force applied to the tube to elicit first movement of the tube vs. the inner diameter of the lumen formed by the tube contacting surface of the securement feature as a percentage of the outer diameter of the tube.

As shown in FIG. 20, for all materials tested there is a “saturation” of the effect of decreasing the diameter of the lumen formed by the arcuate tube contacting surface. In this experiment, the securement feature is being decreased in size relative to the diameter of the tube, which should result in an increase in the securement force applied against the tube when the tube is engaged by the securement feature. However, past about 80% of the diameter, the effect of decreasing the diameter of the tube contacting surface begins to reach a maximum, and likely would begin to decrease as the size of the diameter of the tube contacting surface continues to decrease (as the small tube contacting surface would struggle to make effective contact with the tube due).

While both the silicone material and the PVC material appear to be reaching a maximum at around 80%, and appear to have a linear slope decreasing from about 80% to 100%, the PVC material (for all hardness values) seems to also have a clear increasing slope from 60% to 80%, and it also appears to be linear. A discussion of these slopes is included below.

Difference Between Hardness Values and Materials

Table 3 shows the raw data generated for the experiments looking at the relationship between force and diameter. A one-way ANOVA analysis was performed across the averages of each data set (i.e. Shore A 40 Silicone) shown in Table 3, looking at both differentiation between the sets of data. This analysis showed that there was statistically significant difference between the three different hardness values (40, 50, 60) for the PVC tube alone, with a p value of less than 0.0001. Likewise the data also showed that there may be a statistical difference between the three different hardness values for the silicone tube, with a p value of 0.0590. The one-way ANOVA showed that when comparing all of the three hardness data sets for silicone and PVC, they were not significantly different, with a p value of less than 0.1637.

There may be a variety of reasons for this, the first being there is no difference between the different hardness values for the silicone tube, and then across all the different material tests. However, the data suggests that the 60% diameter data point for silicone had very large data variation (See FIG. 20). The data from the 60% time point for all hardness values was removed. With this data removed, there was a statistically significant difference between the hardness values for silicone, with a p value of less than 0.0075 (The PVC is still significantly different as well, with a p value of 0.0013). However, when performing the one-way ANOVA across all data sets with this data point removed, the results are still not significantly different (p value 0.008). It is likely that this difference is due to the variation that seems to exist in the 60% and 70% variations.

The ANOVA analysis indicated there is a statistically significant difference between the securement force and the material, but not between the materials themselves.

TABLE 3
Raw force data generated for during evaluation of the relationship between
diameter of the tube contacting surface and the securement force

| Diameter | 40 | | | 50 | | | 60 | | |
|---|---|---|---|---|---|---|---|---|---|
| (mm) | Data Set 1 | Data Set 2 | Data Set 3 | Data Set 1 | Data Set 2 | Data Set 3 | Data Set 1 | Data Set 2 | Data Set 3 |

Silicone

| | | | | | | | | | |
|---|---|---|---|---|---|---|---|---|---|
| 4.02 | 1.568 | 1.862 | 1.519 | 2.4402 | 1.5974 | 2.107 | 1.5876 | 1.666 | 1.617 |
| 4.69 | 1.6464 | 1.568 | 1.7934 | 1.8718 | 1.8424 | 1.7738 | 2.3324 | 2.1462 | 2.1952 |
| 5.36 | 1.715 | 1.5974 | 1.568 | 1.6954 | 1.764 | 1.7738 | 2.1168 | 2.1854 | 2.205 |
| 5.69 | 1.3034 | 1.5484 | 1.47 | 1.4602 | 1.2446 | 1.4406 | 1.666 | 1.9796 | 1.8522 |
| 6.03 | 0.8526 | 0.8624 | 1.0192 | 1.3622 | 1.274 | 1.2838 | 1.3818 | 1.4896 | 1.519 |
| 6.37 | 0.637 | 0.588 | 0.7448 | 0.5978 | 0.8428 | 0.9114 | 0.6958 | 0.7252 | 0.7154 |

PVC

| | | | | | | | | | |
|---|---|---|---|---|---|---|---|---|---|
| 4.02 | 0.686 | 0.7742 | 0.6958 | 1.2936 | 1.4308 | 1.3622 | 0.833 | 0.9604 | 0.9212 |
| 4.69 | 1.3426 | 1.4896 | 1.5092 | 1.9698 | 1.7738 | 1.8816 | 1.2152 | 1.1662 | 1.3328 |
| 5.36 | 1.4896 | 1.8522 | 1.715 | 2.058 | 2.0188 | 2.0776 | 1.372 | 1.3622 | 1.3818 |
| 5.69 | 2.058 | 1.9992 | 1.7248 | 2.3128 | 2.2736 | 2.4598 | 1.5974 | 1.4406 | 1.568 |
| 6.03 | 1.6268 | 1.5778 | 1.7542 | 2.0874 | 2.2344 | 1.6464 | 1.323 | 1.421 | 1.4112 |
| 6.37 | 1.3132 | 1.6562 | 1.3328 | 1.4602 | 1.4798 | 1.617 | 1.0094 | 1.47 | 1.1858 |

Analysis of Force/Diameter Relationship

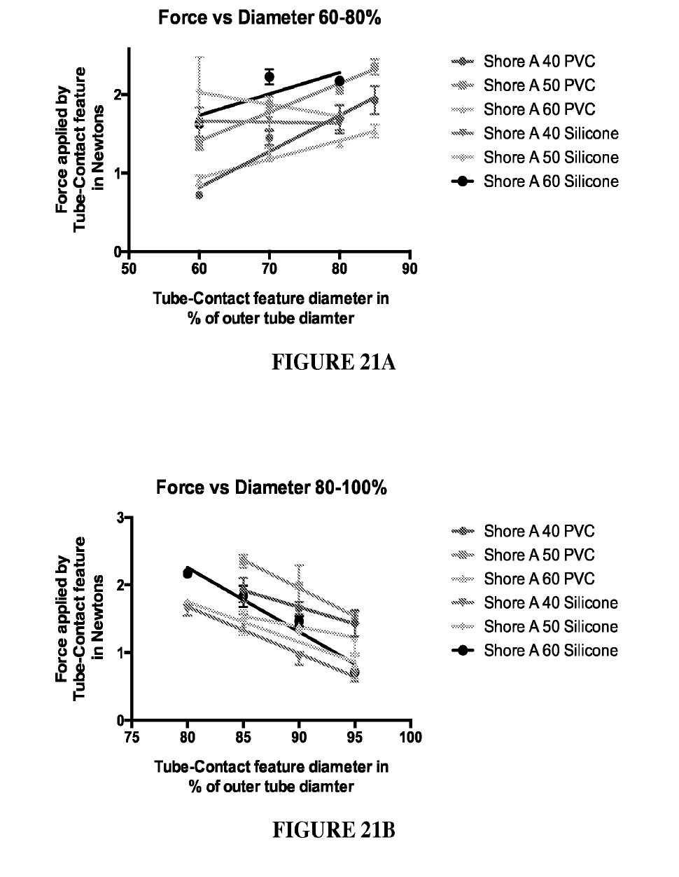

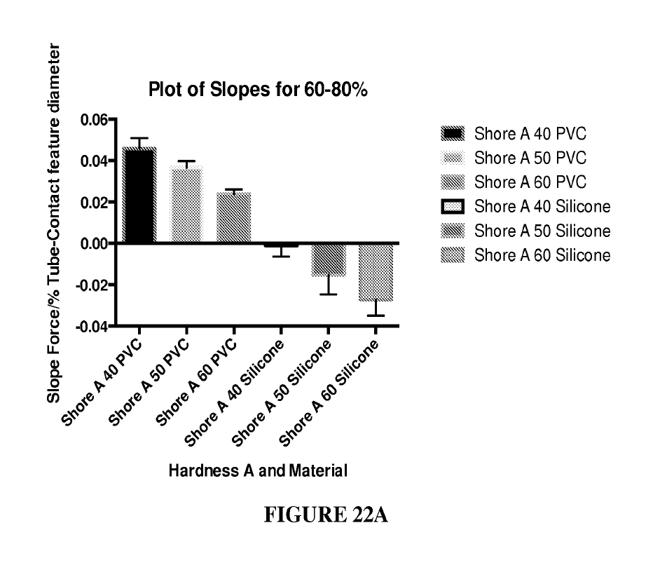

Based on the recognition that for all materials tested a maximum in force was reached when the tube contacting surface diameter was around 80% of the tube diameter, the data was divided into two data sets. The linearity of the data sets was then separately evaluated. The results of this analysis are shown in FIGS. 21A and 21B. The X and Y intercepts for this analysis were not constrained. It was reasoned that at the extreme ends of the range where the tube simply will not fit into the securement feature (smaller percentages) or at the upper end where the tube theoretically does not have any contact (just over 100%), there was not enough data to assume linearity. The slopes of these various curves are shown in Tables 4 and 5.

TABLE 4
Calculated Slopes for Data Sets (Force v. Diameter, 60-80%) for Shore A 40, 50, and 60 materials

| | | | | Shore A 40 | Shore A 50 | Shore A 60 |
|---|---|---|---|---|---|---|
| Best-fit values | Shore A 40 PVC | Shore A 50 PVC | Shore A 60 PVC | Silicone | Silicone | Silicone |
| Slope | 0.04586 ± 0.005029 | 0.03656 ± 0.003184 | 0.02376 ± 0.002277 | −0.001143 ± 0.005095 | −0.01519 ± 0.009487 | 0.02728 ± 0.007585 |
| Y-intercept | −1.937 ± 0.3740 | −0.7873 ± 0.2368 | −0.4900 ± 0.1693 | 1.729 ± 0.3591 | 2.937 ± 0.6686 | 0.09637 ± 0.5345 |
| when X = 0.0 | | | | | | |
| X-intercept | 42.24 | 21.53 | 20.62 | 1512 | 193.4 | −3.533 |
| when Y = 0.0 | | | | | | |
| 1/slope | 21.81 | 27.35 | 42.08 | −874.6 | −65.83 | 36.66 |
| 95% | | | | | | |
| Confidence | | | | | | |
| Intervals | | | | | | |
| Slope | 0.03465 to 0.05706 | 0.02947 to 0.04366 | 0.01869 to 0.02884 | −0.01319 to 0.01091 | −0.03763 to 0.007247 | 0.009339 to 0.04521 |
| Y-intercept | −2.770 to −1.104 | −1.315 to −0.2597 | −0.8672 to −0.1128 | 0.8794 to 2.578 | 1.356 to 4.519 | −1.168 to 1.361 |
| when X = 0.0 | | | | | | |
| X-intercept | 31.71 to 48.78 | 8.792 to 30.19 | 6.018 to 30.15 | 194.7 to +infinity | 119.4 to +infinity | −145.1 to 25.94 |
| when Y = 0.0 | | | | | | |
| Goodness | | | | | | |
| of Fit | | | | | | |
| R square | 0.8926 | 0.9295 | 0.9159 | 0.007141 | 0.2681 | 0.6488 |
| Sy.x | 0.1673 | 0.1059 | 0.07572 | 0.1248 | 0.2324 | 0.1858 |
| Is slope | | | | | | |
| significantly | | | | | | |
| non-zero? | | | | | | |
| F | 83.15 | 131.9 | 109 | 0.05035 | 2.564 | 12.93 |
| DFn, DFd | 1.000, 10.00 | 1.000, 10.00 | 1.000, 10.00 | 1.000, 7.000 | 1.000, 7.000 | 1.000, 7.0 |
| P value | <0.0001 | <0.0001 | <0.0001 | 0.8289 | 0.1534 | 0.0088 |
| Deviation from | Significant | Significant | Significant | Not Significant | Not Significant | Signifi |
| zero? | | | | | | |
| Data | | | | | | |
| Number of X | 4 | 4 | 4 | 3 | 3 | 3 |
| values | | | | | | |
| Maximum | 3 | 3 | 3 | 3 | 3 | 3 |
| number of Y | | | | | | |
| replicates | | | | | | |
| Total number | 12 | 12 | 12 | 9 | 9 | 9 |
| of values | | | | | | |
| Number of | 0 | 0 | 0 | 3 | 3 | 3 |
| missing values | | | | | | |
| Equation | Y = 0.04586 * X − | Y = 0.03656 * X − | Y = 0.02376 * X − | Y = −0.001143 * X + | Y = −0.01519 * X + | Y = 0.02728 * X + |
| | 1.937 | 0.7873 | 0.4900 | 1.729 | 2.937 | 0.09637 |

TABLE 5
Calculated Slopes for Data Sets (Force v. Diameter, 80-100%) for
Shore A 40, 50, and 60 materials

| | Shore A 40 PVC | Shore A 50 PVC | Shore A 60 PVC |
|---|---|---|---|
| Best-fit values | | | |
| Slope | −0.04933 ± 0.01217 | −0.08297 ± 0.01471 | −0.03136 ± 0.01103 |
| Y-intercept when | 6.111 ± 1.097 | 9.420 ± 1.326 | 4.203 ± 0.9940 |
| X = 0.0 | | | |
| X-intercept when | 123.9 | 113.5 | 134 |
| Y = 0.0 | | | |
| 1/slope | −20.27 | −12.05 | −31.89 |
| 95% Confidence | | | |
| Intervals | | | |
| Slope | −0.07812 to −0.02054 | −0.1178 to −0.04818 | −0.05745 to −0.005266 |
| Y-intercept when | 3.517 to 8.705 | 6.285 to 12.56 | 1.852 to 6.554 |
| X = 0.0 | | | |
| X-intercept when | 111.3 to 171.5 | 106.4 to 130.7 | 113.9 to 352.3 |
| Y = 0.0 | | | |
| Goodness of Fit | | | |
| R square | 0.7011 | 0.8196 | 0.5358 |
| Sy.x | 0.1491 | 0.1802 | 0.1351 |
| Is slope significantly | | | |
| non-zero? | | | |
| F | 16.42 | 31.8 | 8.079 |
| DFn, DFd | 1.000, 7.000 | 1.000, 7.000 | 1.000, 7.000 |
| P value | 0.0049 | 0.0008 | 0.025 |
| Deviation from | Significant | Significant | Significant |
| zero? | | | |
| Data | | | |
| Number of X | 3 | 3 | 3 |
| values | | | |
| Maximum | 3 | 3 | 3 |
| number of Y | | | |
| replicates | | | |
| Total number of | 9 | 9 | 9 |
| values | | | |
| Number of | 3 | 3 | 3 |
| missing values | | | |
| Equation | Y = −0.04933 * X + | Y = −0.08297 * X + | Y = −0.03136 * X + |
| | 6.111 | 9.420 | 4.203 |
| | Shore A 40 Silicone | Shore A 50 Silicone | Shore A 60 Silicone |
| Best-fit values | | | |
| Slope | −0.06880 ± 0.005988 | −0.05913 ± 0.007143 | −0.09480 ± 0.007525 |
| Y-intercept when | 7.179 ± 0.5250 | 6.478 ± 0.6263 | 9.839 ± 0.6598 |
| X = 0.0 | | | |
| X-intercept when | 104.3 | 109.6 | 103.8 |
| Y = 0.0 | | | |
| 1/slope | −14.54 | −16.91 | −10.55 |
| 95% Confidence | | | |
| Intervals | | | |
| Slope | −0.08214 to −0.05545 | −0.07504 to −0.04321 | −0.1116 to −0.07803 |
| Y-intercept when | 6.009 to 8.348 | 5.082 to 7.873 | 8.369 to 11.31 |
| X = 0.0 | | | |
| X-intercept when | 101.4 to 108.6 | 104.7 to 117.9 | 101.2 to 107.5 |
| Y = 0.0 | | | |
| Goodness of Fit | | | |
| R square | 0.9296 | 0.8726 | 0.9407 |
| Sy.x | 0.116 | 0.1383 | 0.1457 |
| Is slope significantly | | | |
| non-zero? | | | |
| F | 132 | 68.51 | 158.7 |
| DFn, DFd | 1.000, 10.00 | 1.000, 10.00 | 1.000, 10 |
| P value | <0.0001 | <0.0001 | <0.000 |
| Deviation from | Significant | Significant | Signifi |
| zero? | | | |
| Data | | | |
| Number of X | 4 | 4 | 4 |
| values | | | |
| Maximum | 3 | 3 | 3 |
| number of Y | | | |
| replicates | | | |
| Total number of | 12 | 12 | 12 |
| values | | | |
| Number of | 0 | 0 | 0 |
| missing values | | | |
| Equation | Y = −0.06880 * X + | Y = −0.05913 * X + | Y = −0.09480 * X + |
| | 7.179 | 6.478 | 9.839 |

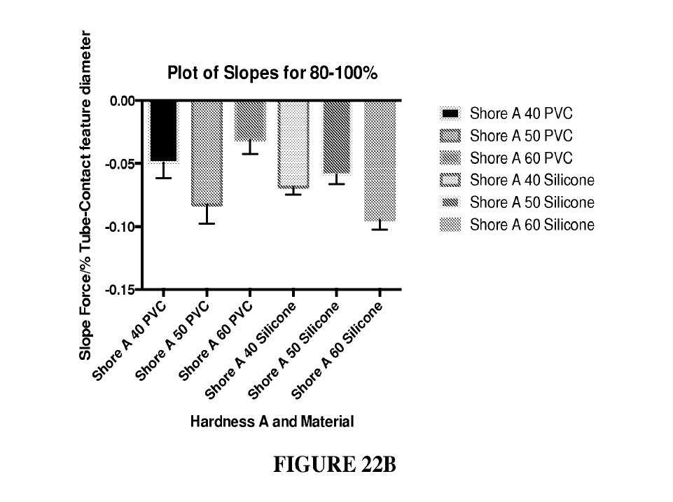

FIGS. 22A and 22B show bar graphs of the data in Tables 4 and 5, respectively.

Taken as a whole the data indicate that that there is a relationship between the diameter of the lumen formed by the tube contacting surface and the amount of securement force the securement feacture is able to exert on a tube placed in contact with the feature. As the relative diameter of the lumen decreases, the amount of securement force applied increases, and this increase is at a first approximation a linear increase in the range of 80% diameter to 100% diameter.

Evaluation of the Relationship Between Berm of the Tube Contacting Surface and Securement Force

Another variable tested was referred to as “berm.” This variable tested the effect on increasing the thickness of the wall supporting the tube contacting surface of each securement feature. As shown in FIG. 23, in certain embodiments the securement feature (identified by the arrow) can take the shape of a “channel” which sits on the top surface of the base. Depending on the radius of the lumen formed by the tube contacting surface of the securement feature and the thickness of the walls of the securement feature, there is a defined amount of the securement feature that is in contact with the top surface of the base. The space on each side of the securement feature can be “filled in” from the top surface of the base and the lateral wall of the securement feature, up to the top of the securement feature. This “fill in”, the “berm,” has a certain radius from the tangent of the contacting the top of the securement feature. This radius was varied, such that as the radius increased, the thickness of the “berm” was increased. It was hypothesized that as the thickness of the berm increased, the amount of force created by the securement feature against the tube would increase. Five different radii were tested: 12 mm, 10 mm, 8 mm, 6 mm, and 4 mm. For all other configurations of the diameter and length tests, the radius was held constant at 4 mm.

Securement Force Related to Berm Radius

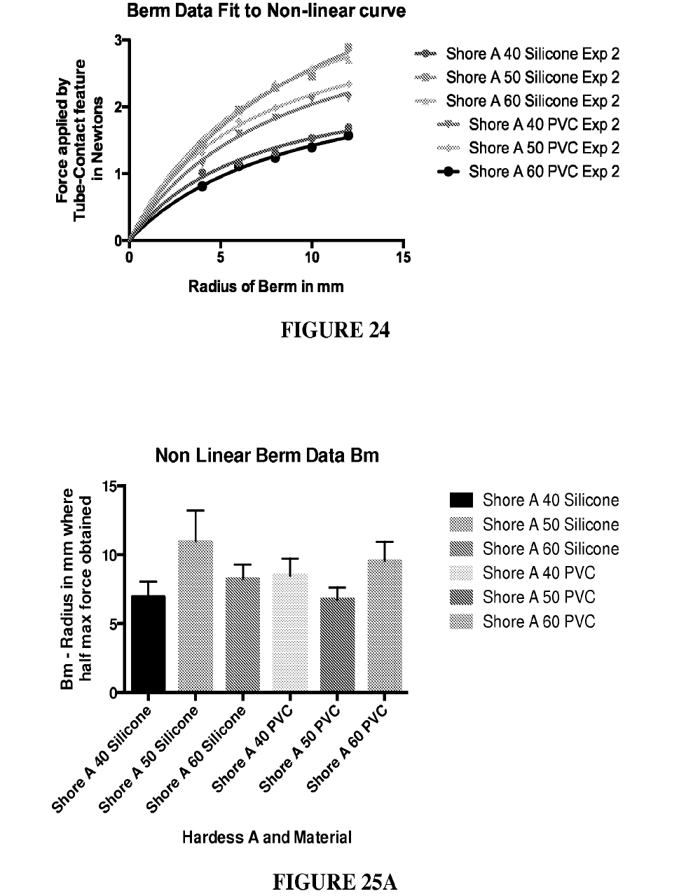

The effect of the radius of the berm supporting the tube contacting surface was tested. The tube contacting surface tested had a length of 8 mm. The diameter of the inner lumen of the tube contact surface was 90% of the outer diameter of the tube (6.03 mm for 20FR), with the berm radius tested at 12 mm, 10 mm, 8 mm, 6 mm, and 4 mm. FIG. 24 shows a plot of the force needed to get the first movement of the tube vs. the berm radius. As shown in FIG. 24, for all materials tested, an increase in the securement force produced by the tube contacting surface was observed as the berm radius (thickness of the berm) was increased. In this case, the berm radius corresponds to the amount of material inhibiting the disfiguration of the tube contacting surface and/or the securement feature. Accordingly, when the berm radius is increased, the tube contacting surface (and by extension the securement feature) applies an increased securement force to the tube when the tube is engage by the securement feature.

Neither the silicone material nor the PVC material appears to exhibit a maximum; rather, they appear to be tapering off, as though approaching a point of saturation. This finding is consistent with the assumption that at some maximum berm radius for a given tube size, the tube contacting surface (and by extension the securement feature) can no longer be deflected (e.g., the tube contacting surface (and by extension the securement feature) would instead deform the tube). Based on this finding, the data was fit using a Michaelis-Menten-type non-linear saturation analysis.

A Michaelis-Menten analysis is typically used in enzyme kinetics and arises from solving the first derivative for the velocity of the reaction based on certain assumptions, which allow the rate of the reaction to be linked to the concentration of the substrate of the reaction. The Michaelis-Menten equation is typically shown as:

V=d[P]/dt=(Vₘₐₓ[S])/(Kₘ+[S]) Eq. 1

where, Vₘₐₓ represents the maximum rate achieved by the system, at maximum (saturating) substrate concentrations. The Michaelis constant, Kₘ, is the substrate concentration at which the reaction rate is half of Vₘₐₓ. This equation was transformed to

F=(Fₘₐₓ[r])/(Bₘ+[r]). Eq. 2

where F is equal to the force, Fₘₐₓ equals the maximum force that can be applied, r equals the radius of the berm, and Bₘ is the “Berm constant.”

A discussion of the calculated parameters is provided below.

Difference Between Hardness Values and Materials

Table 6 shows the raw data generated for the experiments looking at the relationship between force and the berm radius. A one-way ANOVA analysis was performed across the averages of each data set (i.e. Shore A 40 Silicone) shown in Table 6, looking at the difference between the sets of data. This analysis showed that there was statistically significant difference between the three different hardness values (40, 50, 60) for the silicone tube, with a p value of 0.0002 and for the PVC tube, with a p value of less than 0.0001. Likewise the data also showed that there is a statistical difference between the three different hardness values across all data sets for the PVC and the silicone tube, with a p value of less than 0.0001.

TABLE 6
Raw force data generated for during evaluation of the relationship between diameter of the tube contacting surface
and the securement force

| Radius | 40 | | | 50 | | | 60 | | |
|---|---|---|---|---|---|---|---|---|---|
| (mm) | Data Set 1 | Data Set 2 | Data Set 3 | Data Set 1 | Data Set 2 | Data Set 3 | Data Set 1 | Data Set 2 | Data Set 3 |

Silicone

| | | | | | | | | | |
|---|---|---|---|---|---|---|---|---|---|
| 4 | 1.0192 | 0.9996 | 1.029 | 1.3328 | 1.519 | 1.4504 | 1.4014 | 1.4798 | 1.5876 |
| 6 | 1.1172 | 1.2348 | 1.1466 | 1.9012 | 1.8914 | 2.058 | 1.8326 | 1.96 | 2.0188 |
| 8 | 1.274 | 1.3916 | 1.2838 | 2.2344 | 2.303 | 2.3226 | 2.4696 | 2.2638 | 2.2932 |
| 10 | 1.4994 | 1.5778 | 1.5092 | 2.6264 | 2.352 | 2.3814 | 2.548 | 2.6754 | 2.4892 |
| 12 | 1.6464 | 1.7052 | 1.7248 | 2.6362 | 2.9792 | 3.0576 | 2.6656 | 2.7538 | 2.695 |

PVC

| | | | | | | | | | |
|---|---|---|---|---|---|---|---|---|---|
| 4 | 1.1074 | 1.1662 | 1.176 | 1.3132 | 1.4112 | 1.1956 | 0.784 | 0.7938 | 0.8526 |
| 6 | 1.6268 | 1.5974 | 1.568 | 1.7346 | 1.7836 | 1.8522 | 1.1662 | 1.0682 | 1.0976 |
| 8 | 1.9012 | 1.8424 | 1.7542 | 1.9012 | 1.9992 | 2.0384 | 1.2054 | 1.1956 | 1.3132 |
| 10 | 2.156 | 2.1266 | 2.0482 | 2.1756 | 2.0874 | 2.2148 | 1.3818 | 1.323 | 1.47 |
| 12 | 2.2148 | 2.1854 | 2.009 | 2.2638 | 2.3618 | 2.3814 | 1.5288 | 1.5876 | 1.5974 |

The ANOVA analysis indicated there is a statistically significant difference between the securement force and the material, and between the materials themselves.

Analysis of Force/Berm Radius Relationship

A non-linear curve, using a modified Michaelis-Menten-type analysis was used to fit the data shown in Table 6. The results of this analysis are shown in FIG. 24. The Fₘₐₓ was constrained for the fit at less than 10, and the Bₘ was constrained at less than 15, based on visualization of the data. The calculated parameters of these various curves are shown in Table 7.

TABLE 7
Results of Michaelis-Menten-type analysis of data generated for during evaluation of the relationship between
diameter of the tube contacting surface and the securement force

| | Silicone | | | PVC | | |
|---|---|---|---|---|---|---|
| Best-fit values | A 40 | A 50 | A 60 | A 40 | A 50 | A 60 |
| Vmax | 2.592 | 5.392 | 4.644 | 3.766 | 3.657 | 2.776 |
| Km | 6.939 | 10.96 | 8.258 | 8.49 | 6.769 | 9.554 |
| Std. Error | | | | | | |
| Vmax | 0.1893 | 0.6176 | 0.2884 | 0.278 | 0.2087 | 0.2144 |
| Km | 1.108 | 2.242 | 1.033 | 1.247 | 0.8547 | 1.394 |
| 95% Confidence | | | | | | |
| Intervals | | | | | | |
| Vmax | 2.183 to 3.001 | 4.057 to 6.726 | 4.021 to 5.267 | 3.165 to 4.366 | 3.206 to 4.107 | 2.313 to 3.239 |
| Km | 4.545 to 9.332 | 6.119 to 15.00 | 6.027 to 10.49 | 5.796 to 11.18 | 4.923 to 8.616 | 6.542 to 12.57 |
| Goodness of Fit | | | | | | |
| Degrees of Freedom | 13 | 13 | 13 | 13 | 13 | 13 |
| R square | 0.9296 | 0.9339 | 0.9659 | 0.9557 | 0.9579 | 0.9596 |
| Absolute Sum of | 0.06401 | 0.25 | 0.1038 | 0.09087 | 0.0816 | 0.04158 |
| Squares | | | | | | |
| Sy.x | 0.07017 | 0.1387 | 0.08935 | 0.08361 | 0.07923 | 0.05656 |
| Constraints | | | | | | |
| Vmax | Vmax <10.00 | Vmax <10.00 | Vmax <10.00 | Vmax <10.00 | Vmax <10.00 | Vmax <10.00 |
| Km | Km <15.00 | Km <15.00 | Km <15.00 | Km <15.00 | Km <15.00 | Km <15.00 |
| Number of points | 15 | 15 | 15 | 15 | 15 | 15 |
| Analyzed | | | | | | |

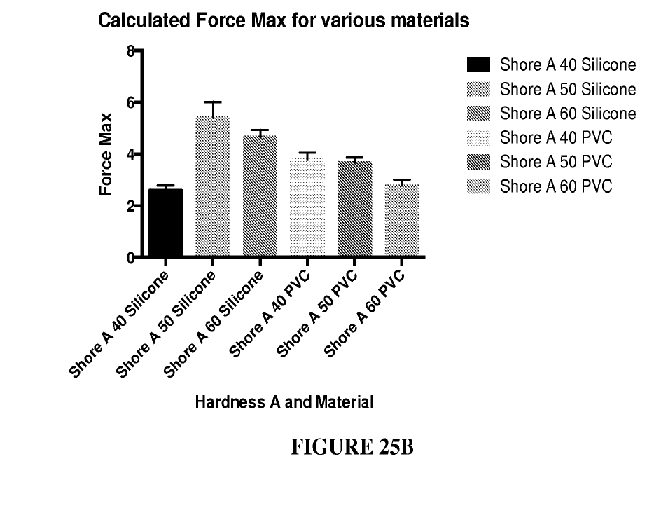

FIG. 25A shows a bar graph of the Bₘ data in Table 7, and FIG. 25B shows the Fₘₐₓ data in Table 7.

The data suggest a relationship between the radius of the berm of the tube contacting surface and the amount of securement force that the tube contacting surface can exert on a tube placed in contact with the securement feature. As the relative radius diameter of the berm increases, the amount of securement force applied to the tube by the tube contacting surface (and by extension the securement feature) increases.

Interestingly, however, the nature of the tube appears to play a role as well. Specifically, the tube contacting surfaces appear to apply a larger securement force against silicone tubes as compared to PVC tubes. In addition, the optimal hardness of the material differs for securement features in contact with silicone tubes as compared to PVC tubes. For example, a silicone A 50 securement feature seems to provide the highest securement force to a silicone tube, while an A40 securement feature seems to provide the highest securement force to a PVC tube. Although, these relatively small differences may be within the error of the experiment.

General Equation for the Securement Force Generation by a Securement Feature

Taking all of the data together, the design of stabilization devices disclosed herein, can be enhanced. The relationship between securement force and the length of the tube contacting surface, securement force and diameter of the tube contacting surface, and securement force and the berm size and hardness, as well as the tube material were used to generate an equation that could be used to estimate the securement force generated by a particular tube contacting surface (and by extension the securement feature). This equation (Eq. 3) can be used, as a first approximation, to estimate the amount of securement force a given tube contacting surface (and by extension the securement feature) will provide, by inputting the length, diameter, and berm radius of the desired securement feature. The various features are adjusted by a constant, 1.19 N and 90% because these were the constants between the various tests. For example, when varying length and berm radius, the diameter was kept at 90%, and so forth.

SF=(1.19N+(TL−8 mm)(SLL(N/mm)))+((90%−TCFD %)(SLD(N/%)))+((Fₘₐₓ*BR)/(Bₘ+BR)−1.19N) Eq. 3

where SF is the securement force (e.g., the frictional force) produced by the tube contacting surface (and by extension the securement feature) on a tube in N; TL is the total length of tube contacting surface in mm; TCFD % is: (inner lumen diameter of the tube contacting surface in mm)/(outer diameter of the tube in mm)*100; Fₘₐₓ=Force max; BR is the berm radius in mm; SLL is the slope of length fit above, SLD is the slope of diameter fit (80-100) above; N is Newtons; and mm=millimeters. Table 8 below includes the various values for Fₘₐₓ (in N), Bₘ (in N/mm), SLL, and SLD, for each hardness and each tube material.

TABLE 8
Values for Fₘₐₓ (in N), Bₘ (in N/mm), SLL, and SLD

| | A 40 | A 50 | A 60 | | A 50 | A 60 |
|---|---|---|---|---|---|---|
| Variable | Silicone | Silicone | Silicone | A 40 PVC | PVC | PVC |
| SLL | 0.1223 | 0.1423 | 0.1174 | 0.1297 | 0.1792 | 0.2043 |
| SLD | 0.04933 | 0.08297 | 0.03136 | 0.06880 | 0.05913 | 0.09480 |
| Fmax | 2.592 | 5.392 | 4.644 | 3.766 | 3.657 | 2.776 |
| br | 6.939 | 10.96 | 8.258 | 8.49 | 6.769 | 9.554 |

Eq. 3 can be used to estimate securement force applied by a tube contacting surface (and by extension a securement feature) as exemplified below. In the case of a tube contacting surface having a length of 10 mm, berm of 6 mm, and a diameter of 90%, and fabricated from Shore A 50 hardness PVC, the calculation for securement force would be:

SF(in N)=(1.19N+((10 mm−8 mm)(0.1792))+((90%−90%)(0.05913))+((3.657*6)/(6.769+6))−1.19N)

SF(in N)=1.5484N+0+0.5284

SF(in N)=2.0768 N.

Eq 3. does not directly take into account the variation associated with each variable and constant identified. However, with the information herein, the Eq. 3 can be adjusted to provide a general direction for design of tube stabilization devices having the characteristics described herein. The relative aspect of the various variables and the amount of force any given tube stabilization device can generate can be assessed. This information can be used to design tube stabilization devices having certain characteristics.

Force Testing and In Vivo Testing of a Chest Tube Stabilization Device

A chest tube device was produced having single securement feature, which was 42 mm long, had a berm radius of 12 mm, and had a diameter of 80% (FIG. 23). This device was made in Shore A 50 hardness Silicone. The ability of the device to grip both silicone and PVC tubes was tested by fixing the device to a vertical support system, by attaching the device with screws through the suture sites along the securement feature portion of the device, as shown in FIG. 23

The device was also tested in a clinical setting with live pigs anesthetized under an IUCUC protocol at the Godley-Snell Animal Center at Clemson University. After being anesthetized, tube thoracostomies were performed, where a number of different stabilization devices were tested, including those, or variants, shown in FIG. 23.

The results of these experiments indicated that the chest tube stabilization devices reduced minor movement of the chest tube at the incision site, relative to one or two purse string sutures or a sandal suture. In addition, major movement, including partial and full dislodgement of the tube were reduced by chest tube stabilizing devices, such as those disclosed herein. Furthermore, chest tube stabilization devices disclosed herein reduced movement relative taping systems in conjunction with purse string or sandal sutures. The in vivo experiments indicated that a variety of tube designs disclosed herein provide at least levels of stabilization of chest drainage tubes sufficient for in vivo use, with reduced movement and no tape or adhesive, relative to existing methods involving purse and sandal sutures along with tape.

The devices and methods of the appended claims are not limited in scope by the specific devices and methods described herein, which are intended as illustrations of a few aspects of the claims. Any devices and methods that are functionally equivalent are intended to fall within the scope of the claims. Various modifications of the devices and methods in addition to those shown and described herein are intended to fall within the scope of the appended claims. Further, while only certain representative devices and method steps disclosed herein are specifically described, other combinations of the devices and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

1. A device for stabilizing a tube in the body of a subject, the device comprising:
(a) a base configured to be secured to a patient comprising a patient contacting surface, an opposing top surface, a proximal end, a distal end, a tube-securing region, and a central reference plane; and
(b) one or more securement features positioned within the tube-securing region, wherein the one or more securement features are configured to reversibly engage the tube such that when the tube is engaged by the one or more securement features, the tube is retained relative to the base.

2. The device of claim 1, wherein the one or more securement features are configured such that when the tube is engaged by the one or more securement features, the one or more securement features apply at least 2 N of securement force to the tube.

3. The device of claim 1, wherein the base further comprises a tube inserting region.

4. The device of claim 1, wherein the base further comprises a plurality of anchor points.

5. The device of claim 4, wherein the plurality of anchor points each individually comprise an eyelet or hook.

6. The device of claim 4, wherein the tube-inserting region comprises a first arm and a second arm that together at least partially define an aperture sized to permit passage of the tube through the aperture from a point above the top surface of the device to a point below the patient contacting surface of the device.

7. The device of claim 1, wherein the one or more securement features are individually chosen from a prong, a clip, a channel, or combinations thereof.

8. The device of claim 1, wherein the device comprises two or more securement features positioned within the tube-securing region.

9. The device of claim 1, wherein the one or more securement features comprise
(a) a first securement feature positioned within the tube-securing region at a first location; and
(b) a second securement feature positioned within the tube-securing region at a second location spaced apart and distal to the first location.

10. The device of claim 9, wherein the first location and the second location are positioned on opposite sides of the central reference plane.

11. The device of claim 9, further comprising a third securement feature positioned within the tube-securing region at a third location spaced apart and distal to the second location.

12. The device of claim 11, wherein the first location, the second location, and the third location are all offset from one another relative to the central reference plane, such that vertically oriented reference planes disposed between the first location and the second location, the first location and the third location, and the second location and the third location when the base is horizontally disposed are not parallel to or coplanar with the central reference plane.

13. The device of claim 1, wherein the one or more securement features comprise
a first prong comprising a first tube contacting surface upwardly projecting from the top surface of the base in the tube-securing region at a first location, and
a second prong comprising a second tube contacting surface upwardly projecting from the top surface of the base in the tube-securing region at a second location spaced apart and distal to the first location.

14. The device of claim 13, further comprising a clip or channel projecting from the top surface of the base in the tube-securing region at a third location spaced apart and distal to the second location.

15. The device of claim 14, wherein the channel comprises a serpentine channel.

16. The device of claim 1, wherein the device is formed of a biocompatible material.
